(19) United States
(12) Patent Application Publication
(10) Pub. No.: US 2025/0177644 A1
(43) Pub. Date: Jun. 5, 2025

(54) Systems and Methods for Expression and Delivery of Proteinaceous Species

(71) Applicant: The Board of Trustees of the Leland Stanford Junior University, Stanford, CA (US)

(72) Inventors: Ada Poon, Redwood City, CA (US); Justin Annes, Palo Alto, CA (US); Rayhan Lal, Stanford, CA (US); Ella A. Thomson, Stanford, CA (US)

(73) Assignee: The Board of Trustees of the Leland Stanford Junior University, Stanford, CA (US)

(21) Appl. No.: 18/970,893

(22) Filed: Dec. 5, 2024

Related U.S. Application Data
(63) Continuation of application No. 63/606,501, filed on Dec. 5, 2023.

(51) Int. Cl. A61M 5/142; A61K 9/50; A61K 35/39; A61K 38/26; A61K 38/28; C12N 15/85

(52) U.S. Cl. CPC A61M 5/14276; A61K 9/5031; A61K 35/39; A61K 38/26; A61K 38/28; C12N 15/85

(57) ABSTRACT

Systems and methods for expression and delivery of proteinaceous species are described. Generally, a system can comprise biological cells within a porous membrane. The biological cells can be utilized to produce proteinaceous species. The biological cells can further store proteinaceous species within secretory granules such that the species can be controllably released upon depolarization of the cell. A secondary force can be provided to release proteinaceous species out of the porous membrane. Proteinaceous species can be expressed from an expression cassette configured to provide controllable release.

### CROSS REFERENCE TO RELATED APPLICATIONS

This application claims benefit of U.S. Prov. Appl. No. 63/606,501, entitled “Systems and Methods for Expression and Delivery of Proteinaceous Species,” filed on Dec. 5, 2023, the disclosure of which is hereby incorporated by reference in its entirety.

### STATEMENT REGARDING FEDERALLY SPONSORED RESEARCH OR DEVELOPMENT

This invention was made with Government support under contracts (TRAINING GRANT) T32 DK007217, DK101530, and DK119955 awarded by the National Institutes of Health. The Government has certain rights in the invention.

### TECHNICAL FIELD

The disclosure is generally directed toward systems and methods for expression and for delivery of proteinaceous species.

### BACKGROUND

Type 1 diabetes (T1D) is a chronic condition in which the pancreas is unable to produce insulin resulting in elevated levels of blood glucose (also referred to as hyperglycemia). Beta cells of the pancreas are responsible for producing insulin by expressing large quantities of insulin peptide and storing the peptides within secretory granulocytes. Upon beta cells receiving a signal that sugar levels are rising within the blood, the membrane of the cells depolarize, resulting in the secretory granules releasing the large quantities of stored insulin peptide into the blood stream. The insulin provides signals to the body to reduce the amount of sugar in the blood to prevent hyperglycemic injury.

There is no cure for T1D and the best treatment options strive to manage blood sugar levels by diet, lifestyle, and controlled delivery of exogenous insulin into the patient's bloodstream. One common method to deliver insulin is the use of an insulin pump that are utilized to maintain a requisite level of insulin levels. A pump can continuously deliver insulin to maintain a basal level of peptide, and in response to carbohydrate consumption resulting in high blood sugar, the pump can deliver a larger bolus of the peptide to acutely reduce the excess sugar in the blood. Regulation of insulin levels is critical to prevent both hyperglycemia and hypoglycemia (i.e., low levels of blood sugar), each of which can cause severe injury to an individual.

### SUMMARY

Several embodiments are directed towards systems and methods for proteinaceous species expression and delivery. In many embodiments, a secretory cell (e.g., a neuroendocrine cell like a beta cell or other cells with controllable secretory pathway) are utilized to express proteinaceous species, such as a peptide or a protein, including (but not limited to) a hormone, a neuropeptide, a cytokine, an immunomodulator, a protein replacement, or an antigen binding species. In several embodiments, a population of secretory cells express proteinaceous species from an endogenous preproinsulin locus or an exogenous preproinsulin expression cassette. In some embodiments, the C-peptide sequence of preproinsulin locus or expression cassette is substituted with a sequence of another proteinaceous species such that is expressed and stored within secretory granules within beta cells and can be controllably released upon depolarization of the cell.

In some embodiments, a population of secretory cells (e.g., neuroendocrine cells) are encapsulated within a porous, immunoprotective membrane. In some embodiments, encapsulated cells produce and secrete an expressed proteinaceous species, such as a peptide or a protein, including (but not limited to) a hormone, a neuropeptide, a cytokine, an immunomodulator, a protein replacement, or an antigen binding species. In some embodiments, in order to improve distribution of secreted proteinaceous species, a secondary force (e.g., pressure) is utilized to drive the secreted proteinaceous species out of a porous, immunoprotective membrane. And in some embodiments, a system for production and delivery of proteinaceous species a population of secretory cells (e.g., neuroendocrine cells) encapsulated within a porous, immunoprotective membrane, the membrane in connection with a means for providing a secondary force, such as a micropump, to distribute proteinaceous species produced and secreted by the cells. In some of these embodiments, the system is utilized as an implantable therapeutic for controllably delivering proteinaceous species to a recipient.

### BRIEF DESCRIPTION OF THE DRAWINGS

The description and claims will be more fully understood with reference to the following figures and data graphs, which are presented as examples and should not be construed as a complete recitation of the scope of the disclosure.

FIGS. 1A, 1B, and 1C provide a schematic of systems for proteinaceous species delivery.

FIG. 2 provides a schematic of a beta cell for production of proteinaceous species.

FIG. 3A provides a schematic of the processing of preproinsulin to yield insulin and C-peptide.

FIG. 3B provides a schematic of the processing of islet amyloid polypeptide.

FIG. 4A provides a schematic of a preproinsulin expression cassette for the expression of transgenes.

FIG. 4B provides a schematic of an islet amyloid polypeptide expression cassette for the expression of transgenes.

FIGS. 5A to 5E provide an illustration of macroencapsulation approaches including islet sheets, subcutaneous approaches with an oxygen source, and our proposed approach incorporating applied pressure. 5A: Diffusion-based methods utilize passive transport for oxygen and insulin, predominantly resulting in designs that are thin and planar due to the limitations of diffusion distances. 5B: Oxygen is either directly supplied to the macroencapsulated islets or generated in situ, which allows for a three-dimensional structure that increases islet surface density. However, insulin transport still primarily depends on diffusion. 5C: Cell stimulation elevates insulin levels within the cell chamber and does not overcome the diffusion limitation. 5D, 5E: The application of brief pressure effectively overcomes diffusion limitations in both macroencapsulation devices, with (5D) and without (5E) enhanced oxygenation.

FIGS. 6A to 6F provide validation of the effect of brief pressure application on insulin transport across a porous membrane through numerical simulations and experimental studies. 6A, 6B: Numerical simulations reveal the prolonged transport time required for diffusion-only transport (6A), contrasting with significantly reduced transport time with the introduction of an 11-kPa pressure (6B). Notably, (6B) provides a zoomed-in view of (6A) during the initial 30 seconds, emphasizing the rapid response facilitated by pressure application. c, Experimental setup for measuring insulin levels within and outside the porous membrane-contained space. 6D, 6E: Empirical measurements of insulin transport under diffusion-only condition (6D) and pressure-driven transport (6E) as compared with theoretical calculations for a membrane with 0.4 μm pore diameter. 6F: Empirical measurement of insulin flux across membranes with pore diameters of 0.2 μm (9.4% porosity), 0.4 μm (12.6% porosity), and 0.8 μm (15.1% porosity), shedding light on the relationship between pore size and insulin transport efficiency. **P<0.01, ****P<0.0001.

FIGS. 7A to 7D provide in vitro validation of repeated insulin bolus delivery from macroencapsulated R7T1 pseudoislets. 7A: Brightfield image of R7T1 pseudoislets. 7B: Experimental setup for measuring insulin levels released from encapsulated R7T1 pseudoislets within and outside the porous membrane-contained space. 7C: Experimental procedure for assessing KCl-stimulated and basal insulin secretion from encapsulated R7T1 pseudoislets with and without applied pressure. Measurements include total insulin released (both inside and outside of encapsulation), and insulin transported to the outside of encapsulation by diffusion and applied pressure for KCl-stimulated and basal R7T1 pseudoislets. 7D: Comparison of insulin flux between diffusion and pressure driven delivery for basal R7T1 pseudoislets over three consecutive 8-hours intervals. **P<0.01, ****P<0.0001.

FIGS. 8A to 8F provide comparative analysis of pressure-driven versus diffusion-based delivery from macroencapsulated mouse islets in WT mice. 8A: An illustration depicting an anesthetized mouse with a cell encapsulation implant and a micropump connected via silicone tubing. PLA, polylactic acid. 8B: Experimental timeline: insulin was administered almost immediately after implantation and a ˜11-kPa pressure was applied for 30 seconds for pressured-treated mice. Blood glucose levels were measured, and blood samples were collected at 0, 15, 30, and 60 minutes after initial insulin dosing. 8C: Change in blood insulin levels observed in mice treated with either diffusion or applied pressure, n=3/group. 8D: Change in blood glucose levels observed in mice treated with either diffusion or applied pressure, n=3/group. 8E: Percentage of dosed insulin remaining in the implant after 90 minutes, n=3/group. 8F: Blood insulin AUC in mice treated with either diffusion or applied pressure, n=3/group. **P<0.01, ***P<0.001, ****P <0.0001.

FIGS. 9A to 9C provide data on restoring euglycemia in diabetic mice via pressure-driven insulin dosing. 9A, 9B, 9C: Changes in plasma insulin and blood glucose levels in mice subjected to either diffusion-based or pressure-driven insulin release from encapsulated islets. Specific islet types used are (9A) mouse islets, (9B) human islets, and (9C) R7T1 pseudoislets. n=5/group. *P<0.05, **P<0.01, ***P<0.001.

FIGS. 10A to 10H provide data and images comparing glycemic control in diabetic mice with repeated pressure-driven insulin delivery at varied durations and intervals. 10A: Schedule for insulin boluses experiments. 10B: Changes in blood glucose levels in mice treated via diffusion, applied pressure, or as sham transplanted controls; n=5 per condition for diffusion and sham, n=6 for pressure. 10C: Changes in blood glucose levels within 60 minutes after administering the first and second doses; n=5 per condition for diffusion and sham, n=6 for pressure. 10D: AUC of change in blood glucose levels for treatments by diffusion, applied pressure, and sham; n=5 per condition for diffusion and sham, n=6 for pressure. 10E: Photograph of the wireless micropump system on a mouse. 10F: Schematic of the wireless micropump system, featuring a high-voltage dual driver (part no. HV861), a Bluetooth transceiver module with an integrated antenna (ANT), a piezoelectric micropump (part no. mp6-liq), and a coin battery (CR1616). 10G: Schedule for basal dosing experiments. 10H: Changes in blood glucose levels in mice treated with Humalog injection or Humalog injection combined with pressure-based dosing; n=4 for pressure, n=5 for sham. ****P<0.0001.

FIGS. 11A and 11B provide data on insulin knockout in R7T1 β cells. CRISPR Cas9 knockout results in decreased expression of insulin in R7T1 β cells evaluated by (11A) immunostaining and (11B) intracellular lysate ELISA.

FIGS. 12A to 12C provide data on engineering the proinsulin vector for glucagon secretion. 12A: Design of the proinsulin glucagon vector targeted for R7T1 enzyme processing. (2B: Engineered proinsulin glucagon vector increased secretion level of glucagon from transfected R7T1 cells but not from transfected HEK293 cells. 12C: KCl stimulation increases glucagon secretion from transfected beta cells.

FIGS. 13A to 13D provide data on validation of constitutive versus secretory release pathways. (3A: A visual representation of the Firefly and renilla luciferase vector insert designs. 13B: A visual illustration of our hypothesis on the secretion of Firefly and renilla luciferase upon KCl stimulation in transfected HEK293 cells and β cells. 13C: Normalized secretion levels of renilla luciferase upon KCl stimulation in transfected HEK293 cells and R7T1 β cells. 13D: Intracellular levels of renilla luciferase in transfected HEK293 cells and R7T1 β cells.

FIG. 14 provides data showing KCl stimulation results in elevated secretion of glucagon and GLP-1 from R7T1 cells transfected with the engineered proinsulin vectors.

### DETAILED DESCRIPTION

Turning now to the drawings and data, systems and methods for proteinaceous species expression and delivery are described. Several embodiments are directed towards the use of a cellular system for production of proteinaceous species, inclusive of proteins, peptides, and any other amino-acid-based polymers that can be expressed and assembled within cells. In many embodiments, the cellular system comprises a population of cells that utilize secretory granules for storage and controlled release of produced proteinaceous species. In some embodiments, the cellular system comprises a population of neuroendocrine cells which utilize secretory granules to controllably release hormones and/or neuropeptides.

Many embodiments are directed towards a proteinaceous species delivery system comprising a population of cells within a membrane-based barrier, wherein the cells are configured to produce and secrete proteinaceous species. In several embodiments, the membrane-based barrier is a composed of a biocompatible material that is immunoprotective to the cells therein, but also comprises a plurality of pores that allow efflux of secreted proteinaceous species. In many embodiments, the proteinaceous species delivery system further comprises a means for providing a secondary force to release secreted proteinaceous species out of the membrane-based barrier. In some implementations of the system, a pump is in connection with the membrane-based barrier to provide a pressure to release secreted proteinaceous species. In several embodiments, the proteinaceous species delivery system is utilized as an implantable device to deliver the secreted proteinaceous species as a therapeutic. In various implementations, the system is controlled in a manner to provide a continuous basal level of and/or a bolus of therapeutic proteinaceous species.

Several embodiments are directed to expression cassettes for production of proteinaceous species. In many embodiments, an expression cassette utilizes sequences for expression of proteinaceous species and storage of expressed proteinaceous species within a secretory granule. In some embodiments, a preproinsulin expression cassette is utilized to express insulin or another proteinaceous species. In some embodiments, the C-peptide sequence of a preproinsulin expression cassette is substituted with a sequence of another proteinaceous species, resulting in that proteinaceous species to be expressed from the cassette. In some embodiments in which the C-peptide sequence of a preproinsulin expression cassette is substituted with a sequence of another proteinaceous species, the B-chain and/or A-chain of insulin mutated or otherwise altered such that disrupts insulin functionality. In some embodiments, an islet amyloid polypeptide (IAPP) expression cassette is utilized to express IAPP or another proteinaceous species. In some embodiments, a propeptide sequence of an IAPP expression cassette is substituted with a sequence of another proteinaceous species, resulting in that proteinaceous species to be expressed from the cassette. In some embodiments, the IAPP sequence of an IAPP expression cassette is substituted with a sequence of another proteinaceous species, resulting in that proteinaceous species to be expressed from the cassette. In several embodiments, a preproinsulin expression cassette and/or an IAPP expression cassette is expressed within a population of secretory cells (e.g., neuroendocrine cells) or other cells having the appropriate machinery to process and controllably release the expressed product.

In many embodiments, the proteinaceous species to be expressed is a hormone, a neuropeptide, a cytokine, an immunomodulator, a protein replacement, or an antigen binding species. In several embodiments, the proteinaceous species to be expressed is nonnative to the cell type, meaning it is not endogenously expressed within the population of cells. In some embodiments, the proteinaceous species to be expressed is nonendogenous by means of sequence alteration, such as expressing an altered insulin peptide from a beta cell.

A derivative or analog of any listed proteinaceous species can be expressed, which is a proteinaceous species having a sequence that is derived from or is analogous to the sequence of the naturally occurring peptide or protein. In some embodiments, a derivative comprises one or more alterations in amino acid sequence and/or peptide processing. Such alterations can be an amino acid polymorphism, amino acid deletion, amino acid inserting, an alteration in posttranslational modification, or an alteration in cleavage. Alterations in posttranslational modifications and cleavage can be achieved by alteration of amino acid sequence or selection/alteration of host cell for expressing and processing the proteinaceous species.

### Systems of Proteinaceous Species Delivery

Several embodiments are directed to systems and methods of proteinaceous species delivery, which can be utilized to improve delivery of the proteinaceous species to an individual. Said delivery can be for a treatment, such as (for example) treating type I or type II diabetes (T1D or T2D). Many other treatments and therapies can be performed, such as delivery of any proteinaceous species described herein. Several species are beneficial for weight control (e.g., In several embodiments, a system of proteinaceous species delivery can utilize a membrane-bound population of cells to express, process, and/or controllably release the proteinaceous species within a patient.

In the example of T1D, current treatments utilize exogenous insulin that is administered via subcutaneous injection or infusion. Individuals living with diabetes require frequent or continuous glucose monitoring, wearing a pump or administering injections, and accounting for carbohydrate ingestion and activity that alters insulin sensitivity. For these individuals, islet transplantation could alleviate patient management burden and cost. One potential approach for islet transplantation is to implant allogeneic islets into the liver via the portal vein. While this approach fosters islet function and viability, and provides insulin independence in the short term, the transplants generally fail due to immunosuppressive toxicity and insufficiency (see, e.g., E. A. Ryan Diabetes. 2001 April; 50 (4): 710-9; and A. M. Shapiro, et al., N Engl J Med. 2006 Sep. 28; 355 (13): 1318-30; the disclosure of which are incorporated herein by reference). Recently, hepatic portal vein infusion of fully differentiated allogeneic stem cell-derived insulin-producing islet cells found success in clinical trials, with two people achieving insulin independence, however, the need for lifetime immunosuppression remains a barrier to widespread deployment (T. W. Reichman, et al., Diabetes. 2023; 72 (Supplement_1): 836-P; the disclosure of which is incorporated herein by reference). Encapsulation of islets with an immunocompatible membrane has been introduced to circumvent the challenge of transplant rejection. Vascularized encapsulated islet transplants have reached the stage of human clinical trials and have been shown to permit direct transfer of nutrient, glucose, and insulin across the membrane such the islet cells can respond to glucose and release insulin. These pouches also have the extra benefit that the islets remain within the membrane, allowing for removal of the transplanted cells. While increased C-peptide levels were observed in patients receiving these implants, the patients did no achieve insulin independence (A. Ramzy, et al., Cell Stem Cell. 2021 Dec. 2; 28 (12): 2047-2061.e5; and A. M. Shapiro, et al, Cell Rep Med. 2021 Dec. 2; 2 (12): 100466; the disclosures of which are incorporated herein by reference).

The islet encapsulation procedure is an actively pursued transplantation approaches for small cell populations. While encapsulation provides a beneficial physical barrier against host immune cell entry, this immunoprotective barrier also impedes the movement of insulin (A. J. Hwa and G. C. Weir, Curr Diab Rep. 2018 Jun. 16; 18 (8): 50; and O. Korsgren, Diabetes. 2017 July; 66 (7): 1748-1754; the disclosures of which are incorporated herein by reference). Relying solely on diffusion for insulin transport is insufficient to achieve physiologic glucose control or insulin independence. For instance, with islets enclosed by a standard porous Polyethylene terephthalate (PET) membrane of area 25 cm², diffusion alone would require approximately 6 hours to transport a human-sized insulin bolus.

Diffusion-based encapsulation is fundamentally incompatible with achieving homeostatic on-demand insulin delivery and physiologic glucose regulation. This critical limitation has been obfuscated by an extensive body of literature that has “validated” islet encapsulation approaches in mice via improved or normalized glucose tolerance, without corresponding rescue of insulin and/or C-peptide release. For example, when one particular immunoisolating device was implanted in diabetic rodents, normal glucose tolerance test results were achieved, but these results were not presented with data on either insulin or C-peptide release (U. Barkai, et al., Cell Transplant. 2013; 22 (8): 1463-76, the disclosure of which is incorporated by reference). Despite the success in rodents, clinical trials of the device in humans fail to demonstrate therapeutic glucose control and insulin independence (B. Ludwig, et al., Proc Natl Acad Sci USA. 2013 Nov. 19; 110 (47): 19054-8; and P. O. Carlsson, et al., Am J Transplant. 2018 July; 18 (7): 1735-1744; the disclosure of which are incorporated herein by reference). Therefore, achieving a normal glucose tolerance test, particularly in rodents, does not affirm an appropriate insulin secretion response necessary for human efficacy.

Reliance on diffusion and rodent glucose tolerance testing has stifled progress in the field of encapsulated islet transplantation. To realize the potential of achieving insulin independence through macroencapsulated cell-based therapy, the current disclosure describes systems that utilize an additional force to increase and control proteinaceous species delivery kinetics (e.g., insulin). Theoretical analysis and experimental findings confirm that the application of a modest applied pressure rapidly and efficiently facilitates peptide flux across encapsulating membranes. This pressure-based dosing method enables sub-minute proteinaceous species release, resembling the behavior of native islets or exogenous infusions. The pressure further allows for controlling repeated bolus and continuous basal species delivery. By harnessing this additional force in device for delivering insulin, euglycemia was acutely restored in mice with complete or near-complete lack of endogenous insulin production within a timeframe unattainable through diffusion alone.

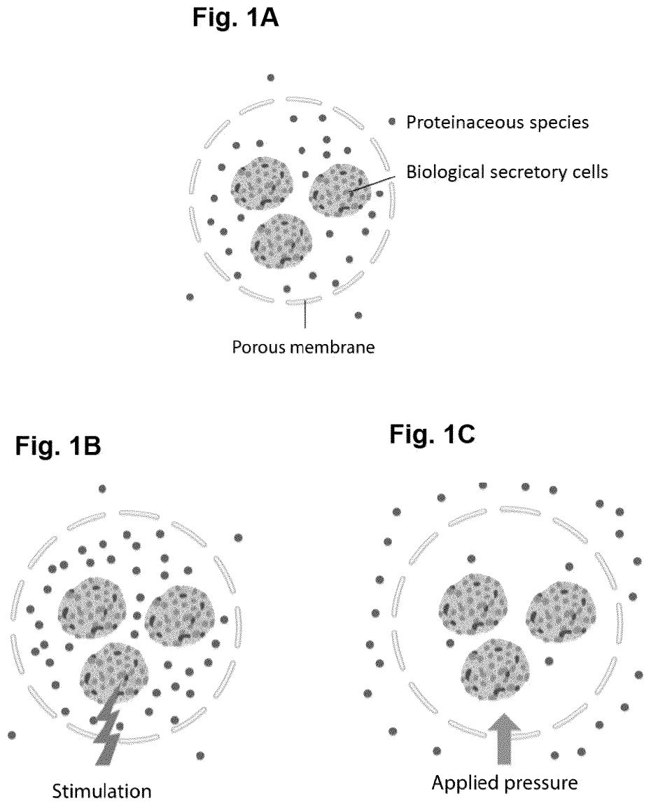

Several embodiments are directed a proteinaceous species delivery system comprising a biological secretory cells within a membrane (FIG. 1A). Biological secretory cells can be utilized to express and secrete a proteinaceous species (e.g., insulin, but see further description of proteinaceous species options). In some embodiments, the biological cells are configured to store proteinaceous species within secretory granules (or similar) such that depolarization of the cells result in release of the proteinaceous species from the cells (FIG. 1B). Depolarization of cells can be stimulated in a control manner, such as (for example) applying a voltage, applying an ionic solution, or stimulating a receptor/ion channel of the cell. In some embodiments, a solution of KCl have a concentration between 10 mM and 100 mM is applied to the cells to induce depolarization and secretion of proteinaceous species, which can be applied via a controlled delivery device.

Any biological cell capable of expressing and secreting a proteinaceous species of interest can be utilized, but especially cells of mammalian origin. In many embodiments, the biological cell is one that is known to store proteinaceous species within secretory vesicles, secretory granules, synaptic vesicles, etc. and secretes the stored proteinaceous species upon a stimulation. Examples of cell types with this capability include (but are not limited to) endocrine cells, acinar cells, neuroendocrine cells, neurons, astrocytes, and granulocytes. Examples of endocrine and neuroendocrine cells include (but are not limited to) alpha (α) cells, beta (β) cells, pancreatic polypeptide (pp) cells, delta (δ) cells, epsilon (ε) cells, enterochromaffin (EC) cells, enterochromaffin-like (ECL) cells, gastrin (G) cells, cholecystokinin (I) cells, K cells, L cells, motilin (Mo) cells, neurotensin (N) cells, secretin(S) cells. Examples of granulocytes include (but not limited to) basophils, eosinophils, neutrophils, and mast cells.

Cells can be derived from a primary source, an immortalized cell line, differentiated from a stem cell, or any other means for deriving a particular cell type. In some embodiments, cells are provided in a microenvironment with multiple cell types, such as (for example) primary tissues and organoids. Examples of primary tissues include pancreatic islets, pulmonary epithelium, gastrointestinal (GI) epithelium, gastric glands, hypothalamus, pituitary gland, thyroid gland, thymus gland, and blood. Endocrine cells can be provided within islets or pseudo islets. Neuroendocrine cells can be derived from and/or maintained as neuroendocrine tumors (NETs).

Biological secretory cells can be maintained within the membrane in a manner that allows for continued proteinaceous species production. Generally, cells can be maintained in solution with factors that maintain and/or promote cell vitality and proteinaceous species production. Precise growth and maintenance conditions will depend on the cell type, as is understood in the art.

A proteinaceous species is a polymer of amino acids, such as a peptide or a protein. In several embodiments, a proteinaceous species is capable of expressed from a sequence nucleic acids via translation by ribosomes. Any proteinaceous species can be configured to be secreted via stimulation. In several embodiments, endogenous proteinaceous species are secreted upon stimulation. For example, beta cells can be utilized to secrete insulin, alpha cells can be utilized to secrete glucagon, delta cells can be utilized to secrete somatostatin, acinar cells can be utilized to secrete digestive enzymes, neuroendocrine cells can be utilized to secrete neurotensin, etc. In some embodiments, a biological secretory cell is configured to secrete a nonendogenous proteinaceous species, which is any species that is not naturally expressed in the cell, which may include derivatives of endogenous species that have had their sequence altered. In some embodiments, a biological secretory cell is configured to secrete a transgenic proteinaceous species. For example, an expression cassette comprising preproinsulin gene that is modified by substituting the C-peptide with a transgene can be utilized within a beta cell, resulting in a transgenic beta cell configured to secrete the transgene proteinaceous species product via its secretory granules. In another example, an expression cassette comprising an IAPP gene that is modified by substituting a propeptide and/or IAPP with a transgene can be utilized within a beta cell, resulting in a transgenic beta cell configured to secrete the transgene proteinaceous species product via its secretory granules. For more description on expression of transgenes via a preproinsulin expression cassette or an IAPP expression cassette, see ensuing sections and appendices. Other cellular secretory systems and expression cassettes can be coopted in a similar manner to express and secrete transgenic proteinaceous species.

In many embodiments, the proteinaceous species to be expressed is a hormone, a neuropeptide, a cytokine, an immunomodulator, a protein replacement, or an antigen binding species. In several embodiments, the proteinaceous species to be expressed is nonnative to the cell type, meaning it is not endogenously expressed within the population of cells. In some embodiments, the proteinaceous species to be expressed is nonendogenous by means of sequence alteration, such as expressing an altered insulin peptide from a beta cell. A number of examples of proteinaceous species can be expressed. Examples of hormones an neuropeptides that can be expressed include (but are not limited to) insulin (INS), glucagon (GCG), gastric inhibitory polypeptide (GIP), glucagon-like peptide 1 (GLP-1), glucagon-like peptide 2 (GLP-2), leptin (LEP), pancreatic polypeptide (PP), somatostatin (SST), substance P (SP), ghrelin appetite hormone (GAH), gastrin (GAST), peptide YY (PYY), neuropeptide Y (NPY), cholecystokinin (CKK), angiotensin (Ang), neurotensin (NTS), islet amyloid polypeptide (IAPP), motilin (MLN), secretin (Sct), vasopressin (AVP), vasoactive intestinal peptide (VIP), calcitonin (CT), growth hormone (GH), insulin-like growth factor 1 (IGF-1), follicle-stimulating hormone (FSH), oxytocin (OT), thyroid-stimulating hormone (TSH), gonadotropin-releasing hormone 1 (GnRH1), gonadotropin-releasing hormone 2 (GnRH2), dynorphins (Dyn), endorphins, endomorphins (EM), nociceptin (NC). Examples of cytokines that can be expressed include (but are not limited to) type 1 interferons (IFNs), interferon gamma (IFNG), tumor necrosis factor alpha (TNF-α), interleukin 2 (IL-2), interleukin 7 (IL-7), interleukin 12 (IL-12), and interleukin 21 (IL-21). Examples of immunomodulators that can be expressed include (but are not limited to) programmed cell death protein 1 antibody (anti-PD-1), programmed death ligand 1 antibody (anti-PD-L1), and tumor necrosis factor alpha antibody (anti-TNF-α). Examples of proteins for protein replacement that can be expressed include (but are not limited to) factor VIII (FVIII), cystic fibrosis transmembrane conductance regulator (CFTR), and enzymes for lysosomal storage disease (e.g., α-glucocerebrosidase or α-galactosidase). Examples of antigen binding proteinaceous species include immunomodulators, receptor tyrosine-protein kinase erbB-2 antibody (anti-HER2); vascular endothelial growth factor antibody (anti-VEGF), cluster of differentiation 20 antibody (anti-CD20), and pathogen neutralizing antibodies.

A derivative or analog of any listed proteinaceous species can be expressed, which is a proteinaceous species having a sequence that is derived from or is analogous to the sequence of the naturally occurring peptide or protein. In some embodiments, a derivative comprises one or more alterations in amino acid sequence and/or peptide processing. Such alterations can be an amino acid polymorphism, amino acid deletion, amino acid inserting, an alteration in posttranslational modification, or an alteration in cleavage. Alterations in posttranslational modifications and cleavage can be achieved by alteration of amino acid sequence or selection/alteration of host cell for expressing and processing the proteinaceous species. Many alterations will modulate the function and/or stability of the peptide or protein as compared to the naturally occurring form. Modulation can be any modification to the function or stability of the species, such as (for example) improved function, reduced function, altered function (e.g., selective activation), loss of function, improved half-life stability, decreased half-life stability, altered biodistribution, altered targeting, increased expression, increased translation, decreased expression, decreased translation, altered immunogenicity, and altered processing. Furthermore, a derivative or analog of any antibody or T-cell receptor listed can be an antigen binding proteinaceous species that is expressed. Herein, an antigen binding proteinaceous species is a proteinaceous species comprising one or more complementarity-determining regions (CDRs) derived from or analogous to an antibody or T-cell receptor, and is configured to maintain binding affinity for its antigen. Any other portion of antigen binding proteinaceous species other than the one or more CDRs can be majorly altered in any way as long as binding affinity for its antigen for its antigen is maintained.

Several embodiments are directed towards the use of a porous membrane to encapsulate the cells. Various types of membranes can be utilized. Generally, the membrane comprises a plurality of pores with a porosity capable of allowing efflux of secreted proteinaceous species but not allowing biological cells to traverse therethrough. The membrane can also be biocompatible and well tolerated as an implant within a recipient. In some embodiments, the membrane is immunoprotective and is thus capable of preventing an immune response against the biological cells therein (e.g., mitigates signaling of immune cells).

The membrane can be composed of a synthetic or organically derived material. Examples of materials that can be utilized include (but are not limited to) poly(ethylene) (PE), poly(ethylene terephthalate) (PET), ultra-high molecular weight poly(ethylene) (UHMWPE), thermoplastic poly(urethane) (TPU), expanded poly(tetrafluoroethylene) (ePTFE), and alginate; which can be combined and/or fluorinated.

The dimensions and characteristics of the membrane can be configured to support encapsulation of cells and efflux of the proteinaceous species. In some implementations, the membrane is configured to have a size that allows for retrievability after implantation. In some embodiments, the area of the membrane is at least 5 cm², which is large enough allow retrievability. In some implementations, the thickness and porosity of the membrane are configured to allow efflux of the proteinaceous species. In some embodiments, a membrane has a thickness between 1 μm and 100 μm. In some embodiments, a membrane has a porosity between 5% and 25%. In some embodiments, the average length of the pore diameter is between 5 μm and 2 mm, in which larger pores can be utilized for larger microtissues, organoids, islets, or scaffolded cells.

In many embodiments, a proteinaceous species delivery system comprises a means for providing a force to facilitate efflux of the proteinaceous species out of a membrane (FIG. 1C). Any force for facilitating the efflux of the proteinaceous species out of a membrane can be utilized. In some embodiments, an applied pressure is provided to facilitate efflux of the proteinaceous species. It has been found via theoretical computation and experimentation that an applied pressure of ˜11 kPa will result in in can efflux greater than 90% of a bolus of insulin peptide within twenty seconds (see Examples Section). Accordingly, in some embodiments, an applied pressure of between 2 kPa and 25 kPa can be utilized, which can be adjusted based on parameters of the membrane and size of the proteinaceous species.

In various embodiments, an applied pressure is provided to facilitate efflux of the proteinaceous species, the average applied pressure being between 2 kPa and 4 kPa, between 3 kPa and 5 kPa, between 4 kPa and 6 kPa, between 5 kPa and 7 kPa, between 6 kPa and 8 kPa, between 7 kPa and 9 kPa, between 8 kPa and 10 kPa, between 9 kPa and 11 kPa, between 10 kPa and 12 kPa, between 11 kPa and 13 kPa, between 12 kPa and 14 kPa, between 13 kPa and 15 kPa, between 14 kPa and 16 kPa, between 15 kPa and 17 kPa, between 16 kPa and 18 kPa, between 17 kPa and 18 kPa, between 18 kPa and 19 kPa, between 19 kPa and 20 kPa, between 20 kPa and 22 kPa, between 21 kPa and 23 kPa, between 22 kPa and 24 kPa, or between 23 kPa and 25 kPa.

Any means for applying a pressure can be utilized. In some embodiments, a micropump is utilized, such as (for example) a piezoelectric micropump. The micropump can be in connection with the membrane such that a pressure can be applied. In some implementations, the micropump is connected such that a direct pressure is applied to the internal compartment of the membrane. In some implementations, a laminar flow along and adjacent to the membrane is applied to draw the peptides out of the membrane.

A pump system can comprise a variety to ensure adequate function. For instance, a pump system can comprise a power source (e.g., battery), a pump driver to draw power from the power source and send it to the pump, a controller system for controlling the power and timing of the pump, and tubing for connecting the pump to the membrane. In some implementations, a controller system utilizes wireless communication such that control inputs can be provided by a remote device. In some implementations, the pump system comprises a fluid reservoir to provide a source of fluid (e.g., saline). In some implementations, the pump draws from local source of fluid (e.g., plasma). In some implementations, the pump is configured to provide an acute applied pressure to facilitate efflux of a bolus of proteinaceous species. In some implementations, the pump is configured to additionally or alternatively provide a continual or continuous applied pressure to facilitate efflux of a continual or continuous basal amount of proteinaceous species.

Many embodiments are directed towards a proteinaceous species delivery system being utilized as an implant. In several embodiments, an implant can comprise a set of one or more membrane-encapsulated cell systems that is in connection with a micropump system. An implant can be configured in any manner to allow for proper use. Accordingly, an implant can comprise a housing with the set of one or more membrane-encapsulated cell systems and micropump system therein. The implant can be configured such that when installed on a recipient, proteinaceous species can be released in a manner to reach its intended target site. For example, an implant for the delivery of insulin can be configured to subcutaneously release insulin, allowing released insulin to enter into the blood stream.

Several embodiments are directed to the use of a proteinaceous species delivery system. Generally, a proteinaceous species delivery system can be provided. In some embodiments, a proteinaceous species delivery system is utilized as an implant and is installed within a recipient, such as (for example) within the vascular system, the lymph system, the cerebral spinal fluid system, or locally at a site to be treated. The method can allow a population biological secretory cells to express and process proteinaceous species. In some embodiments, the method allows the biological cells store proteinaceous species within secretory granules (or similar). The method can stimulate secretion of proteinaceous species from the biological cells, which can be achieved by inducing depolarization of the cells. The method can further provide a force to distribute the proteinaceous species through the membrane and into tissue of the recipient. In some implementations, the method continually or continuously releases the proteinaceous species (e.g., to provide a basal amount of proteinaceous species). In some implementations, the method acutely releases the proteinaceous species (e.g., to provide a bolus of proteinaceous species). In some implementations, the population of biological cells are able to respond to endogenous signals. For example, a system for releasing insulin can encapsulate islets that allow glucose to pass membrane, and the cells can respond to glucose levels within the circulatory system. A micropump can also be in connection with a sensor (e.g., glucose sensor or insulin sensor) and provide an appropriate pressure level when detecting an analyte, such as high levels of glucose or excreted insulin within the encapsulated membrane. In some embodiments, the sensor, when activated, can send a signal to the pump to perform a program of providing pressure. For example, a pump can be programmed to perform a bolus pump followed by low-level consistent pressure.

### Systems for Production of Proteinaceous Species

Several embodiments are directed towards systems that allow for production and controlled secretion of proteinaceous species. Recombinantly produced proteins constitute a substantial portion of newly developed and approved drugs, and therapeutics. While bacteria have historically served as cell factories for drug production, there has been a notable shift in recent years towards utilizing mammalian cell lines, such as HEK293 cells, Hela cells, plasma cells, and Chinese Hamster Ovary (CHO) cells. Despite this transition, therapeutic production primarily occurs in in vitro environments using large bioreactors. Prevalent mammalian cell lines used for recombinant protein production lack granular protein storage and a secretory protein release pathway. As a result, these cell lines exhibit constitutive release of produced proteins and lack the machinery necessary to release a substantial amount of protein within a short time frame. This presents challenges, particularly for proteins susceptible to rapid degradation, like glucagon and GLP-1, or those necessitating the release of a substantial protein quantity within a precise time frame, like glucagon for hypoglycemia recovery.

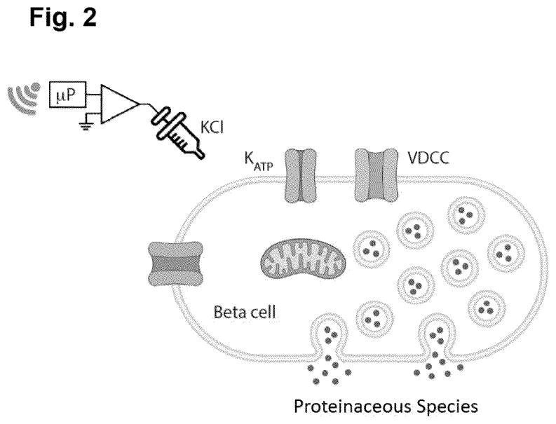

Here, secretory cells (e.g., beta cells) are utilized as an on-demand cell factory for expressed proteinaceous species. Endocrine and neuroendocrine cells stand out as an ideal target cell line due to their possession of vesicle protein storage and a regulated secretory protein release pathway. As noted, other cells can be utilized as well, such as acinar cells, neurons, astrocytes, and granulocytes. The proteinaceous species, once translated and processed, are stored within vesicles within the cells (FIG. 2). The process of membrane depolarization, induced by stimuli such as glucose, KCl (FIG. 2), or electrical signals, propels the exocytosis of the stored proteinaceous species from the cell. This intricate mechanism facilitates the prompt and controlled release of a substantial quantity of stored proteinaceous species, responding swiftly to demand within a sub-minute to minute time frame. In the examples provided herein, the use of beta cells were utilized for the synthesis of glucagon and GLP-1, highlighting their controlled release along the secretory release pathway of beta cells. Notably, although FIG. 2 depicts the exocytosis of proteinaceous species from beta cells by means of KCl stimulation, it is to be understood that any of the cells described herein can be manipulated to release a proteinaceous species upon stimulus by coopting the secretory signals of the endogenously stored and exocytosed species.

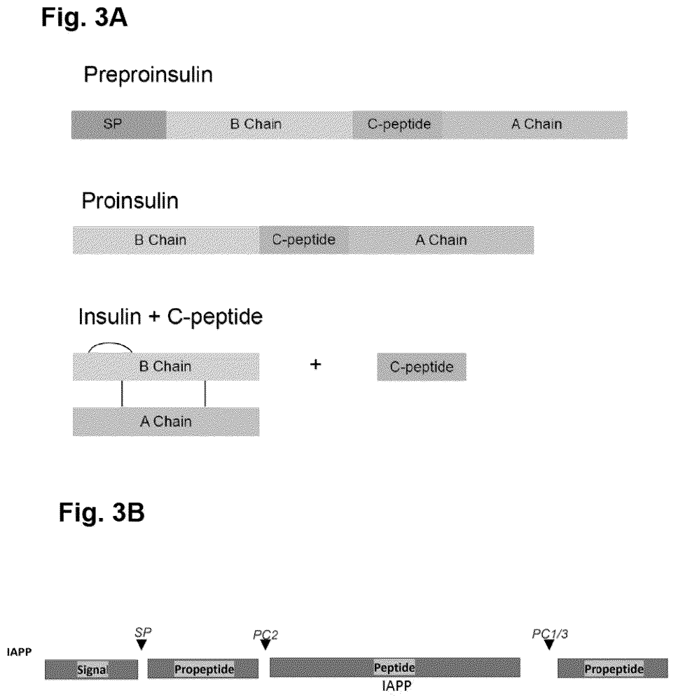

For description of principle, beta cells and secretion of insulin and IAPP are described. But any secretory cells described herein can be utilized to express and process a proteinaceous species of choice by coopting the cell's endogenous processing system. Beta cells natively produce and process insulin. Insulin is initially translated as preproinsulin, comprising a signal peptide (SP), A-chain, B-chain and C-peptide (FIG. 3A). The signal peptide guides preproinsulin into the endoplasmic reticulum for processing. The interaction of the signal peptide with cytosolic ribonucleoprotein signal recognition particles enables translocation into the lumen across the rough endoplasmic reticulum. Subsequently, the signal peptide is cleaved by signal peptidase, resulting in the formation of proinsulin (FIG. 3A). Proinsulin undergoes folding, forming three disulfide bonds, and is then transported to the Golgi apparatus. Within the Golgi apparatus, it enters secretory vesicles, where enzymes cleave proinsulin into mature insulin and C-peptide (FIG. 3A). Both mature insulin, composed of the A-chain and B-chain linked by disulfide bonds, and C-peptide are stored in vesicles within the cell. In a similar manner, beta cells also use similar processes to yield islet amyloid polypeptide (IAPP).

To direct the release of a proteinaceous species of interest along the secretory pathway of beta cells, expression vectors were designed to be processed in a similar manner to insulin or IAPP. In one implementation, a propreinsulin expression vector was designed in which the sequence of the C-peptide portion of proinsulin was substituted with a transgene for expressing a proteinacous species. In another implementation, the sequence of the propeptides and IAPP were each substituted with a transgene. To prevent insulin signaling from these expression systems, mutations were introduced to the sequences of the A-chain and the B-chain of the propreinsulin expression vector to ensure that the transgene product is co-secreted with a non-bioactive form of insulin.

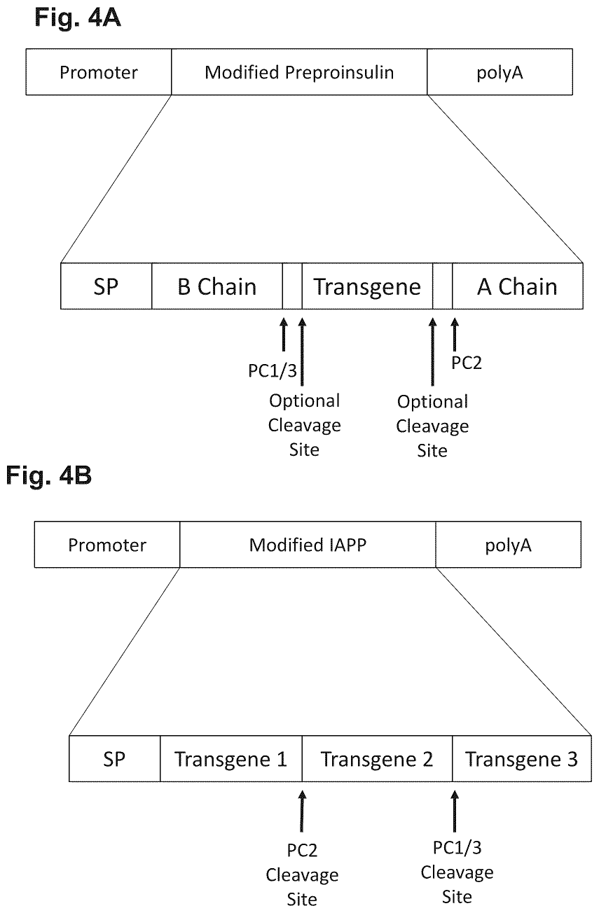

Provided in FIGS. 4A and 4B are examples of expression constructs in accordance with several embodiments. In FIG. 4A, the translated portion comprises a modified preproinsulin DNA sequence, which comprises a signal peptide, a B chain, an A chain, and a transgene substituted in place of the C-peptide. In FIG. 4B, the translated portion comprises a modified prepro-IAPP DNA sequence, which comprises a signal peptide, transgene 1 in place of a propeptide, transgene 2 in place of IAPP, and transgene 2 in place of another propeptide. To allow for processing, sequences that signal for cleavage by prohormone convertase 1/3 (PC 1/3) or prohormone convertase 2 (PC2) can be added flanking the sequence of the transgene (e.g., within 0 to 10 amino acids of the transgene). The optional cleavage sites can be provided near to the transgene to prevent unnecessary amino acid overhangs but in some instances may not be immediately adjacent to allow for further processing by carboxypeptidases and the amidation by the enzyme PAM. Flanking the 5′-end of the preproinsulin cDNA can be a promoter such that the promoter is operatively linked to promote expression. Likewise, flanking the 3′-end of the preproinsulin cDNA is a poly-A signal such that the poly-A signal is operatively linked to add poly-A tail to a transcribed preproinsulin RNA molecule.

Although particular modified preproinsulin and modified IAPP expression cassettes are depicted in FIGS. 4A and 4B, various other combinations and modifications are contemplated for use in a beta cell. Generally, an expression cassette can include a signal peptide (SP) and any number of transgenes, each separated by a PC1/3 or PC 2 cleavage site. Accordingly, an expression cassette can express several transgenes (e.g., any number between 1 and 10). Further, an expression cassette can be configured to yield a particular stoichiometric ratio. For example, to yield a stoichiometric ratio of 1:3, an expression cassette can include two transgenes in which one of the transgenes is repeated 3 times, each separated by a PC1/3 or PC 2 cleavage site. Stoichiometric ratios of transgenes may be configured to improve therapeutic design that includes multiple therapeutic transgene products having different characteristics such as therapeutic efficiency, half-life, etc.

The expression construct can further comprise other expression elements that may enhance expression, including (but not limited to) WPRE sequence, Kozak sequence, enhancers, and one or more introns (e.g., beta-globin intron).

In some embodiments, one or both of the A chain and B chain is mutated to yield a biologically inactive insulin peptide product. Any mutation that inactivates, yet maintains proper codon sequence, can be utilized.

In some embodiments, a modified preproinsulin or modified IAPP DNA sequence further comprises a selectable marker (also referred to as a reporter gene) that is expressed. The selectable marker can include a DNA sequence for expressing the selectable marker product and can be linked to the modified preproinsulin via an internal ribosome entry site (IRES), a self-cleaving peptide (e.g., 2A peptide), or a signal to be cleaved by the machinery of the beta cell (e.g., PC 1/3 peptide or PC 2 peptide). Selectable markers include (but are not limited to) fluorescent proteins (e.g., GFP, eGFP, RFP, etc.), antibiotic resistance markers (e.g., beta-lactamase, neomycin, hygromycin, etc.), and luciferase (e.g., firefly luciferase, Renilla luciferase, etc.). Typically, a selectable marker is provided downstream of the modified preproinsulin sequence, but can be provided upstream in some implementations.

For expression cassettes to be utilized within a beta cell, various embodiments are directed to substituting the sequence of the C-peptide, an A chain, a B-chain, a prepeptide, and/or an IAPP with a sequence of transgene. Any nonendogenous gene for expression of a proteinaceous species can be substituted, including peptides and proteins. For example, a C-peptide can be substituted with a non-native C-peptide gene, an IAPP can be substituted with a non-native IAPP gene, etc. The substituted gene can provide expression of any of the proteinaceous species described herein, a derivate thereof, or an analog thereof. In some embodiments, the transgene is codon optimized for human expression, especially if the transgene is of non-human origin. In some embodiments, the transgene comprises a sequence for a protein tag. Protein tags that can be utilized include (but are not limited to) His-tag, Myc-tag, HA-tag, FLAG-tag, VSV-tag, T7-tag, biotinylation site, Fc-tag, glutathione-S-transferase-tag, SNAP-tag, CLIP-tag, etc.

In many embodiments, an expression cassette is configured to express a hormone, a neuropeptide, a cytokine, an immunomodulator, a protein replacement, or an antigen binding species. In several embodiments, the proteinaceous species to be expressed is nonnative to the cell type, meaning it is not endogenously expressed within the population of cells. In some embodiments, the gene product to be expressed is nonendogenous by means of sequence alteration, such as expressing an altered insulin peptide within a beta cell. A number of examples of gene products can be expressed. Examples of hormones an neuropeptides that can be expressed include (but are not limited to) insulin (INS), glucagon (GCG), gastric inhibitory polypeptide (GIP), glucagon-like peptide 1 (GLP1), glucagon-like peptide 2 (GLP2), leptin (LEP), pancreatic polypeptide (PP), incretin, somatostatin (SST), substance P (SP), ghrelin appetite hormone (GAH), gastrin (GAST), peptide YY (PYY), neuropeptide Y (NPY), cholecystokinin (CKK), angiotensin (Ang), neurotensin (NTS), islet amyloid polypeptide (IAPP), motilin (MLN), secretin (Sct), vasopressin (AVP), vasoactive intestinal peptide (VIP), melanin concentrating hormone (MCH), adrenocorticotropic hormone (ACTH), adropin, atrial natriuretic peptide (ANP), calcitonin (CT), growth hormone (GH), insulin-like growth factor 1 (IGF-1), follicle-stimulating hormone (FSH), luteinizing hormone (LH), melanocyte-stimulating hormone (MSH), oxytocin (OT), parathyroid hormone (PH), prolactin (PRL), renin, thyroid-stimulating hormone (TSH), thyrotropin-releasing hormone (TRH), gonadotropin-releasing hormone 1 (GnRH1), gonadotropin-releasing hormone 2 (GnRH2), corticotropin-releasing hormone (CRH), enkephalin (ENK), resistin (RETN), dynorphins (Dyn), endorphins, endomorphins (EM), nociceptin (NC). Examples of cytokines that can be expressed include (but are not limited to) type 1 interferons (IFNs), interferon gamma (IFNG), tumor necrosis factor alpha (TNF-α), interleukin 2 (IL-2), interleukin 7 (IL-7), interleukin 12 (IL-12), and interleukin 21 (IL-21). Examples of immunomodulators that can be expressed include (but are not limited to) programmed cell death protein 1 antibody (anti-PD-1), programmed death ligand 1 antibody (anti-PD-L1), and tumor necrosis factor alpha antibody (anti-TNF-α). Examples of proteins for protein replacement that can be expressed include (but are not limited to) factor VIII (FVIII), cystic fibrosis transmembrane conductance regulator (CFTR), and enzymes for lysosomal storage disease (e.g., α-glucocerebrosidase or α-galactosidase). Examples of antigen binding proteinaceous species include immunomodulators, receptor tyrosine-protein kinase erbB-2 antibody (anti-HER2); vascular endothelial growth factor antibody (anti-VEGF), cluster of differentiation 20 antibody (anti-CD20), and pathogen neutralizing antibodies.

In some embodiments, any particular nucleotide or amino acid sequence may have a sequence that is modified from its native sequence. Alterations in sequence may be intentionally employed for any number of reasons, such as (for example) codon optimization, improved translation, ability to distinguish from an endogenously expressed gene product, derivatives or analogs that have enhanced activity, derivatives or analogs that are more stable, derivatives or analogs that are immunoevasive, or derivatives or analogs that evade proteases. Accordingly, in some embodiments, a proteinaceous species has an amino acid sequence that is altered from its native sequence and/or endogenously expressed sequence in the cell used for expression and processing. In various embodiments, the amino acid sequence of an expressed proteinaceous species has between 50% and 60% homology to its native sequence, has between 60% and 70% homology with its native sequence, has between 50% and 60% homology with its native sequence, has between 70% and 80% homology with its native sequence, has between 80% and 90% homology with its native sequence, has between 90% and 95% homology with its native sequence, has between 95% and 99% homology with its native sequence, has between than 99% and 100% homology with its native sequence, or has 100% homology with its native sequence. In addition, a proteinaceous species can have a sequence that is differentiated from a cognate proteinaceous species endogenously expressed in the cell. In various embodiments, the amino acid sequence of an expressed proteinaceous has between 50% and 60% homology with its cognate endogenously expressed in the cell, has between 60% and 70% homology with its cognate endogenously expressed in the cell, has between 70% and 80% homology with its cognate endogenously expressed in the cell, has between 80% and 90% homology with its cognate endogenously expressed in the cell, has between 90% and 95% homology with its cognate endogenously expressed in the cell, has between 95% and 99% homology with its cognate endogenously expressed in the cell, has between than 99% and 100% homology with its cognate endogenously expressed in the cell, or has 100% homology with its cognate endogenously expressed in the cell.

To express a proteinaceous species of the disclosure, nucleic acids encoding proteinaceous species are inserted into expression vectors such that the coding sequence is operatively linked to transcriptional and translational regulatory sequences. The term “regulatory sequence” refers to nucleic acid sequences that effect the expression and translation of RNA coding sequences to which they are operably linked. Such regulatory sequences may include a promoter, transcription termination, a splice cassette, translation initiation codon, and/or translation termination. The term “operably linked” refers to a juxtaposition of a regulatory sequence with a coding sequence permitting them to function in their intended manner. Examples of regulatory sequences for gene expression in eukaryotic host cells include (but are not limited to) mammalian regulatory sequences CMV-promoter, SV40-promoter, RSV-promoter, SFFV-promoter, insulin-promoter, CMV-enhancer, SV40-enhancer and a globin intron. Regulatory elements may also include transcription termination signals, such as (for example) the SV40 poly-A site or the tk-poly-A site, typically operably linked downstream of the expression sequence.

Typically, expression cassettes can be propagated in host cells by including sequences for plasmid or virus maintenance and for cloning and expression of exogenous nucleotide sequences. Such sequences typically include one or more of the following operatively linked regulatory sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Several embodiments are directed towards the expression of a modified preproinsulin cassette and/or modified IAPP cassette within a beta cell. Expression within a beta cell allows for processing of the prepro-proteinaceous product to yield one or more cleaved a transgene products. In some embodiments that include sequence of an insulin product, the product is a biologically inactive insulin product. Beta cells have an advantage that products that are expressed with a signal peptide can be stored within a secretory granule, which can be controllably and/or responsively secreted by the beta cell. Transgene products can be harvested quickly after secretion, which can allow for higher yields of products with a short half-life or is otherwise difficult to harvest in a constitutive producing cell line. Alternatively, beta cells are utilized in an implant system to deliver the transgene product to the recipient.

Any beta cell capable of being propagated in vitro and manipulated to express a modified preproinsulin and/or a modified IAPP cassette can be utilized to produce proteinaceous products. Examples of beta cells that can be utilized is human EndoC-betaH1 cell line, mouse R7T1 beta cell line, mouse Min6 beta cell line, rat Ins-1 beta cell line, primary beta cells, and stem-cell derived beta cells. Primary and stem-cell derived beta cells can be of any mammalian species, but especially of human, mouse, or rat origin.

In some embodiments, beta cells for expressing a proteinaceous product have one or more endogenous genes disrupted such that they do not express and/or yield a biologically active products. In particular, endogenous genes that yield products that are stored and secreted within secretory granules may be disrupted, such as (for example) insulin gene(s) or IAPP gene(s). Any mechanism for disrupting endogenous gene(s) can be utilized, such as (for example) site-directed mutagenesis via a CRISPR-Cas system.

Beta cells can express an expression cassette in any manner to confer expression of proteinaceous products. Accordingly, an expression cassette can be expressed from an episomal vector or from a sequence integrated within the host genome, which can be transiently or stably expressed. The vector can be a plasmid, viral vector, or any other suitable vector. Cassettes can be integrated with a host genome via viral integration (e.g., lentivirus), site-directed mutagenesis integration (e.g., CRISPR-Cas system), or any other suitable integration technique.

Other secretory cells and modified transgene expression cassettes can be utilized. Examples include (but are not limited to) alpha cells and a modified GCG expression cassette, pp cells and a modified PP expression cassette, delta cells and a modified somatostatin expression cassette, epsilon cells and a modified ghrelin expression cassette, G cells and a modified gastrin expression cassette, I cells and a modified CCK expression cassette, K cells and a modified GIP expression cassette, L cells and a modified GLP-1, modified GLP-2, or a modified peptide YY expression cassette, Mo cells and a modified MLN expression cassette, N cells and a modified NTS expression cassette, and S cells and a modified Sct expression cassette. For each modified expression cassette, a transgene can be substituted within the preprocessed gene product such that it is expressed, processed, and stored within secretory granules.

Suitable methods for nucleic acid delivery to effect episomal or integrate transgene expression can include virtually any method by which a nucleic acid (e.g., DNA or RNA) can be introduced into a beta cell, as described herein or as would be known to one of ordinary skill in the art. Such methods include, but are not limited to, direct delivery of nucleic acids such as by injection, by microinjection, by electroporation, by chemical transfection, by liposome transfection; etc. Other methods include viral transduction, such as gene transfer by lentiviral or retroviral transduction. In some embodiments, recombinant expression cassettes are contacted with host cells of a recipient to induce expression. An expression construct encoding an expression sequence can be injected, transfected, transduced or infected into a recipient according to a variety of methods known in the art.

Numerous expression systems can be utilized to propagate an expression cassette. Commercially and widely available systems include in but are not limited to bacterial, mammalian, yeast, and insect cell systems.

Several embodiments are directed towards methods to yield transgene products. In some embodiments, a method comprises providing a secretory cell system, such as described herein. The method can further comprise contacting a modified transgene expression cassette with the secretory cell. For example, a modified preproinsulin cassette and/or a modified prepro-IAPP cassette can be contacted with a beta cell such that the cassette can effect transgene expression. The method can further comprise allowing a beta cell to generate secretory granules comprising the transgene product. The method can further comprise stimulating the secretory cell such that secretes the transgene product out of the beta cell. The method can further comprise collecting the secreted transgene product, which can be further purified and/or prepared for downstream applications. Alternatively, a population of secretory cells with a modified cassette can be encapsulated within a membrane of an implant system as described herein. The implant system can be combined with a micropump or other means for providing pressure to distribute secreted proteinaceous species across the membrane. The implant system can be used for treatment of a recipient in need responsive and/or controlled release of the proteinaceous species.

Generally, a treatment method can comprise the installation of transplant system comprising a membrane-encapsulated population of secretory cells into a recipient to be treated by controlled and/or responsive release of a proteinaceous species in which the population of secretory cells express, process and store the proteinaceous species within secretory granules and exocytose the proteinaceous species upon stimulation. The transplant system can further comprise a micropump or other means for providing pressure that is utilized to distribute the secreted proteinaceous species across the membrane and into tissue of the recipient. A micropump can further be controlled and/or responsive such that the pressure is supplied concurrently or after the secretion of the proteinaceous species. The micropump can be in connection with a sensor for detecting a signal, and upon detection of the signal the micropump is activated to provide pressure. Examples of signals a sensor could detect include (but are not limited to) the stimulus that induces exocytosis of the proteinaceous species, the secreted proteinaceous species within the membrane, or membrane potential current resulting from depolarization of the secretory cell.

A vast number of medical conditions can be treated by controlled and/or responsive administration of proteinaceous species. Examples of treatments include (but are not limited to): INS for treatment of hyperglycemia, T1D, T2D excessive fat deposit (e.g., obesity), or metabolic syndrome; GCG for treatment of hypoglycemia, T1D, T2D, or metabolic syndrome; GIP for treatment of excessive fat deposit (e.g., obesity), appetite suppression, or promoting weight loss; GLP-1 for treatment of excessive fat deposit (e.g., obesity), appetite suppression, or promoting weight loss; GLP-2 for treatment of gastrointestinal distress, short bowel syndrome, inflammatory bowel disease, or chemotherapeutically-induced mucositis; leptin is used to treat congenital leptin deficiency, lipodystrophy, hypoleptinemia, hypertriglyceridemia, insulin resistance, hepatic steatosis, or excessive fat deposit (e.g., obesity); PP for treatment of hyperglycemia, T1D, T2D excessive fat deposit (e.g., obesity); SST for treatment of ulcers, gastritis, esophageal varices, neuroendocrine tumors, acromegaly, or diabetic retinopathy; SP for treatment of pathogenic infection; GAH for treatment of cachexia, anorexia, or to increase weight gain; GAST for treatment to improve nutrient digestion; PYY for treatment of excessive fat deposit (e.g., obesity) or to reduce appetite; NPY for treatment of anxiety, depression, neurodegeneration, to induce neuroprotection, or to increase appetite; CCK for treatment of T2D, excessive fat deposit (e.g., obesity), or anxiety; Ang is used to treat hypotension and nasal congestion; NTS can be used for treatment of schizophrenia; IAPP can be used for treatment of hyperglycemia, T1D, T2D, and excessive fat deposit (e.g., obesity); MLN can be used for treatment of delayed gastric emptying or gastroparesis; SCT can be used to treat autism, or schizophrenia; AVP can be used as treatment for diabetes insipidus or to reduce stomach bloat; VIP can be used as treatment for pulmonary hypertension, chronic obstructive pulmonary disease, rheumatoid arthritis, or T2D; CT can be used for treatment of Paget's disease of the bone or bone loss with osteoporosis; GH can be used for treatment of GH deficiency, chronic kidney disease, Turner syndrome, or Prader-Willi syndrome; IGF-1 can be used for treatment of kids that are not growing on schedule, T2D or metabolic syndrome; FSH can be used for treatment of infertility in women; OT can be used for treatment to improve lactation, reduce heavy bleeding after birth, or improve sexual bonding; TSH can be used for treatment of hyperthyroidism; GnRH1 or GnRH2 can be used for treatment of endometriosis, precocious puberty, heavy menstrual bleeding, premenstrual dysphoric disorder, or transgender hormone therapy; Dyn can be used for treatment of pain or mood regulation; endorphins can be used for treatment of pain, stress, or to improve sense of well-being; EM can be used for the treatment of pain or inflammation; nociception can be used for the treatment of chronic pain, anxiety, or alcohol dependence; IFNs can be used for the treatment of cancer and pathogenic infection; IFNG can be used for the treatment of chronic granulomatous disease or severe malignant osteopetrosis; IL-2 can be used for the treatment of cancer; IL-7 can be used for the treatment of lymphopenia, chronic viral infection, or hematopoietic stem cell transplantation; IL-12 can be used for the treatment of cancer; IL-21 can be used for the treatment of cancer; anti-PD-1 and anti-PD-L1 can be used for the treatment of cancer; anti-TNF-α can be used for the treatment of rheumatoid arthritis, plaque psoriasis, ankylosing spondylitis, ulcerative colitis, or Crohn's disease; factor VIII can be used for the treatment of hemophilia; cystic fibrosis transmembrane conductance regulator can be used for the treatment of cystic fibrosis; α-glucocerebrosidase can be used for the treatment of Gaucher disease; α-galactosidase can be used for the treatment of Fabry disease; anti-HER2 can be used for the treatment of breast cancer; anti-VEGF can be used for diabetic retinopathy, macular edema, age-related macular degeneration, or cancer; anti-CD20 can be used for the treatment of B-cell cancers, rheumatoid arthritis, systemic lupus erythematosus, and multiple sclerosis; and pathogen neutralizing antibodies can be used for the treatment of pathogenic infection.

### EXAMPLES

Biological data enable and support the systems and methods for proteinaceous species expression and delivery. In the following section, schematics and data are provided that detail systems for improving peptide delivery and systems for on-demand peptide production.

Enhancing Therapeutic Insulin Transport from Macroencapsulated Islets Using Sub-Minute Pressure at Physiological Levels

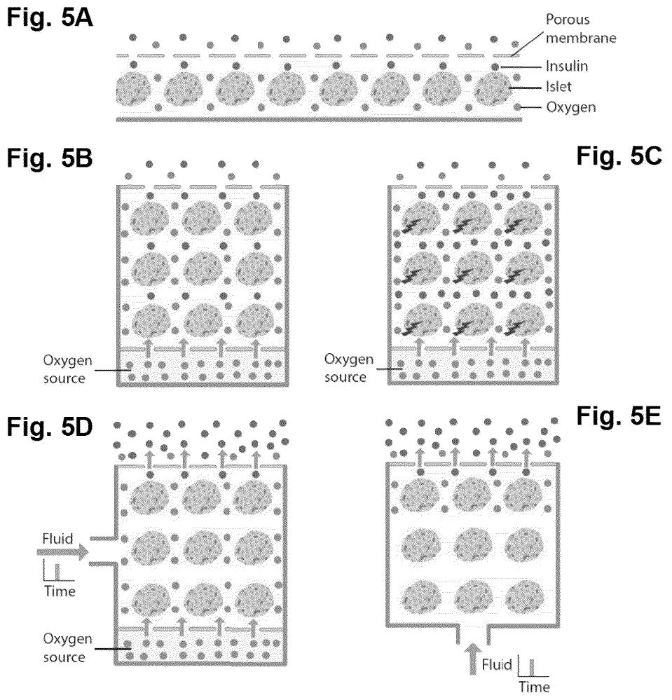

To enhance islet oxygenation while maintaining immunoisolation, the physiological pressure difference between arteries and veins is utilized. This process facilitates convective transport of oxygen and essential nutrients. High islet viability, at a density of 5,700 IEQ/cm2, has been demonstrated in a porcine model. However, these systems are associated with substantial surgical risks. To mitigate risk and enable subcutaneous transplantation, pump-based extravascular methods are being investigated. Among these, the bAir device, which utilizes an exogeneous oxygen supply has advanced to clinical trials but has not successfully demonstrated therapeutic glucose control or insulin independence. Additionally, other techniques supporting subcutaneous transplantation with enhanced oxygenation include prevascularization of graft sites and in situ oxygen generation methods such as hydration of solid peroxide, recycling of carbon dioxide, electrolysis, and microalga-based photosynthesis. In these subcutaneous approaches, insulin transport predominantly relies on diffusion as illustrated in FIGS. 5A and 5B. For instance, with islets encapsulated within a PET membrane spanning 25 cm², the islet surface density must be increased by at least 28 times per the Edmonton protocol. If the islet volume density remains constant, the thickness of the cell chamber must also increase 28-fold. Consequently, the diffusion time for insulin would increase proportionally, meaning diffusion alone would require approximately ˜5 hours to transport 90% of the insulin secreted within the cell chamber. Furthermore, cell stimulation only leads to an increase in insulin levels within the cell chamber and is not able to overcome the diffusion limitation, as illustrated in FIG. 5C.

Thus far, macroencapsulation strategies have primarily focused on enhancing cell viability to achieve high islet surface density. However, the discussion often overlooks the diffusion limitation for insulin transport across the immunoisolating membrane. This gap poses significant challenges in achieving effective on-demand insulin delivery and maintaining physiological glucose regulation—both essential for homeostasis. Regrettably, this crucial limitation has been overshadowed by extensive research that has “validated” islet encapsulation methods in mice, demonstrating improved or normalized glucose tolerance but without a corresponding increase in insulin and/or C-peptide release. These studies often neglect the well-documented effect of glucose effectiveness—the ability of glucose to manage its own disposal and hepatic glucose output. This effect is more pronounced in rodents than in humans44-46, with the magnitude of this difference being at least twice as significant. In streptozotocin (STZ)-induced diabetic rodents, simply implanting an insulin pellet, that supplies only basal insulin, along with glucose effectiveness, suffices to normalize glucose tolerance tests. Similarly, the bAir device, when implanted in diabetic rodents, resulted in normal glucose tolerance test outcomes, yet data reflecting the release of insulin or C-peptide are not provided. Achieving normal glucose tolerance test results, particularly in rodents, does not affirm an adequate insulin transport needed for human efficacy.

To harness the clinical potential of macroencapsulated cell-based therapy, an additional driving force is required to enhance and regulate insulin transport across the immunoisolating membrane, as illustrated in FIGS. 5D and 5E. To enable subcutaneous transplantation, we propose an extravascular results show that applying pressure for a sub-minute duration at a level akin to physiological diastolic pump-based method to surpass the barriers of diffusion. Our mathematical and experimental blood pressure is sufficient to rapidly and effectively transport insulin through encapsulating membranes. This method replicates the delivery dynamics of native islets or external insulin infusions and facilitates repeated bolus insulin delivery. By harnessing a second driving force, we acutely restored euglycemia in mice with complete or near-complete lack of endogenous insulin production within a timeframe unattainable through diffusion alone.

### Order-of-Magnitude Analysis of Pressure Duration and Magnitude on Insulin Transport Across Porous Membranes

To describe how applied pressure is a feasible second driving force, we consider the simultaneous effect of pressure (p) and concentration (cₛ) gradients on the transport of insulin across a porous membrane. The insulin (solute) flux, Jₛ, is the combined effect of the solute diffusional flow and the driving force from the volumetric solvent flux, Jᵥ. The solute diffusional flow −Dₛ∇cₛ is driven by the insulin concentration gradient where Dₛ is the insulin diffusion coefficient. The volumetric solvent flux is a pressure-driven flow; this flow in turn carries the insulin to flow through the membrane resulting in an insulin flux of Jᵥcₛ=−Lₚh∇ₚ·cₛ where Lₚ is the filtration coefficient and h is the membrane thickness. Here, we are interested in finding a simple expression for the ratio of the insulin flux with and without the applied pressure in steady state to comprehend the multiplicative effect of applied pressure relative to diffusion alone for insulin transport. This expression is given by

$$\frac{{P}_{e}}{1 - {e}^{- {P}_{e}}} \cdot \frac{{c}_{s , in} - {c}_{s , out} {e}^{- {P}_{e}}}{{c}_{s , in} - {c}_{s , out}} \approx {P}_{e} \qquad ( Eq . 1 )$$

where c_{s,in} and c_{s,out} are the insulin concentration inside and outside the encapsulation respectively. In the ratio, Pₑ is the axial Peclet number defined by

$${P}_{e} = \frac{{L}_{p} h Δ p}{{D}_{s}} .$$

When Pₑ is large, the ratio in Eq. 1 approximates to Pₑ; that is, the Peclet number alone reveals the multiplicative effect of applied pressure relative to diffusion for insulin transport.

For insulin transport where membrane pore radius (rₚ) is much greater than molecular radius of insulin (rₛ), we apply the Hagen-Poiseuille Law and Strokes-Einstein Equation to obtain

$${P}_{e} = \frac{3 π {r}_{s}}{4 {k}_{B} T} \cdot {r}_{p}^{2} \cdot Δ p = 7.36 \times {10}^{11} \cdot {r}_{p}^{2} \cdot Δ p \qquad ( Eq . 2 )$$

where k_B is the Boltzmann constant and T is the absolute temperature. Here, we showed that the multiplicative effect of applied pressure compared to diffusion alone on insulin transport is primarily determined by two key factors: the membrane's pore radius and the magnitude of the applied pressure. For typical insulin transport membranes (pore radius ˜sub-μm), this ratio approximates to 0.1Δp. That is, a 11-kPa applied pressure (roughly equivalent to normal diastolic blood pressure in humans) will increase steady-state insulin flux by ˜3 orders of magnitude. During transient transport, this multiplicative effect in insulin flux translates into a scaling of release time. An 11-kPa applied pressure takes 3 orders of magnitude less time to transport the same amount of insulin for a given membrane area. Furthermore, a 11-kPa pressure may be provided by a range of micropumps36-38 with dimensions less than a few centimeters in size, suitable for implantation in humans.
Numerical Simulation and Measurement of Insulin Flux Through an Encapsulation Membrane with and without a Small Pressure Gradient

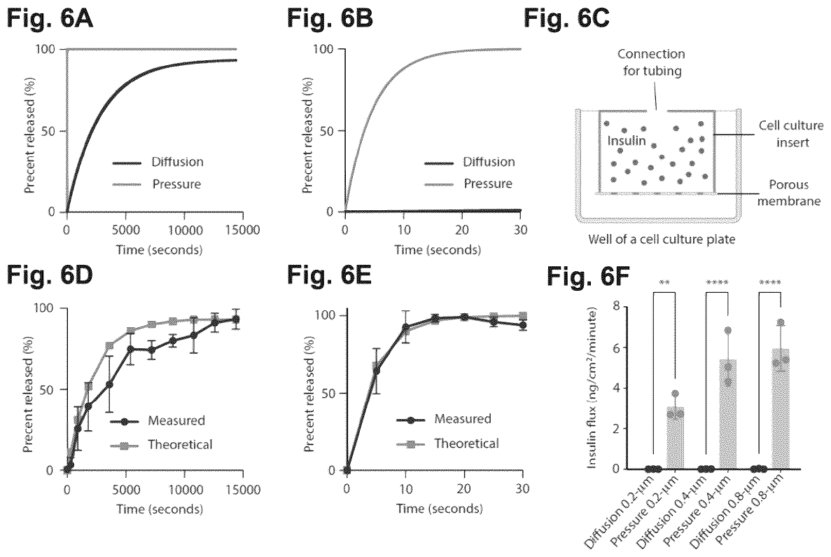

We performed numerical simulations of insulin transport across a membrane area in the range of square centimeters. Our findings revealed that relying solely on diffusion required over 2 hours to transport over 90% of encapsulated insulin (FIG. 6A). However, with the application of an 11-kPa pressure, transport time was markedly reduced to approximately 10 seconds (FIG. 6B), thereby affirming conclusions from our earlier theoretical analysis. Although our theoretical analysis used 10 kPa as an order-of-magnitude reference, we opted for 11 kPa in our simulations and experiments to more closely approximate the normal human diastolic blood pressure of about 10.7 kPa. This pressure was generated by our custom-designed piezoelectric micropump system, which effectively mimics normal human diastolic conditions.

Subsequently, we conducted experimental studies using a porous PET membrane with a 0.4 μm pore diameter and 12% porosity, commonly used for encapsulation. These experiments were conducted in a 12-well format, using encapsulated cell culture inserts (FIG. 6C). Pressure was applied using a piezoelectric micropump, maintaining an average pressure of ˜11 kPa at the with no observed backflow. The empirical findings corroborated the simulation results (FIGS. 6D and 6E), consistent across a range of pore sizes (FIG. 6F). For pore sizes ranging from 0.2 to 0.8 μm (equivalent to rp in Eq 2 in the range of 0.1 to 0.4 μm), the introduction of brief pressure enhanced flux by an average of approximately 400 times compared to diffusion alone. Therefore, a brief application of pressure akin to normal human diastolic blood pressure markedly shortened the time required for transporting encapsulated insulin, from several hours via diffusion alone to less than a minute.

In Vitro Validation of Repeated Insulin Bolus Delivery from Encapsulated R7T1 Pseudoislets and Human Islet

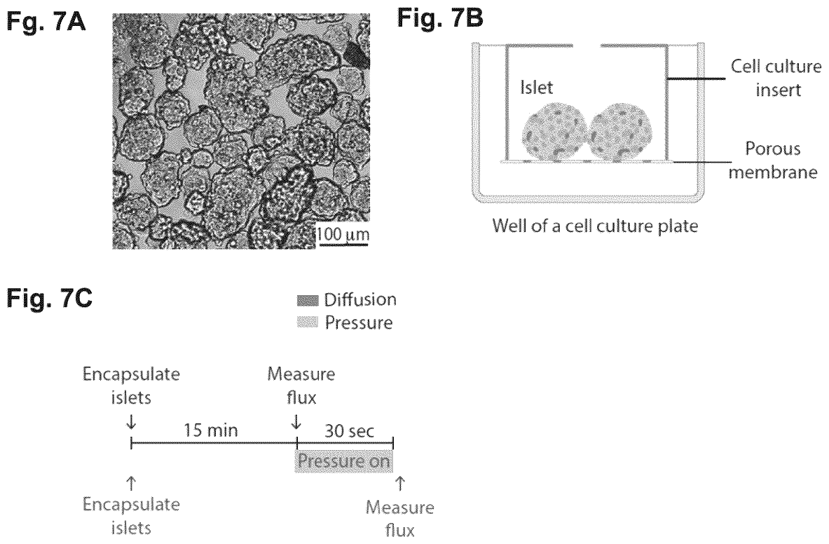

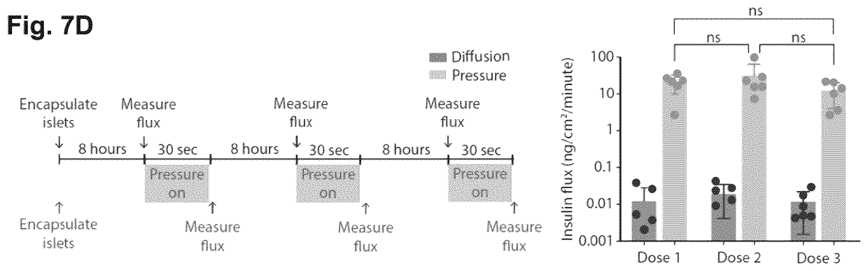

Building upon the aforementioned findings, we hypothesized that (1) the encapsulation membrane hinders diffusion-dependent insulin delivery from secretagogue-stimulated β-cells; and (2) the application of a brief pressure can generate insulin boluses independently of cell stimulation, such as glucose-stimulated insulin secretion (GSIS) and KCl-triggered insulin secretion. To investigate these hypotheses, we used the setup illustrated in FIG. 5E. We utilized the R7T1 β-cell pseudoislets (FIG. 7A) and characterized them by measuring insulin secretion under both proliferating and growth arrested conditions, as well as assessing the expression of ZnT8, insulin, and PDX1. Furthermore, we examined how the number of R7T1 pseudoislets affects insulin secretion, validating an approximately linear correlation between pseudoislet count and insulin release. In our experiments, approximately 800 pseudoislets (˜685 IEQ) were encapsulated within porous PET (200 μL, 2284 IEQ/cm2) using the experimental setup shown in FIG. 7B.

To test the first hypothesis, encapsulated pseudoislets were exposed to either basal [5 mM glucose] or stimulatory [40 mM KCl] culture conditions. As anticipated, a 15-minute KCl treatment significantly increased total secreted insulin (p<0.0001; FIG. 7C). We selected the 15-minute time point to approximate the recommended pre-meal insulin bolus injection time in individuals with T1D. When solely relying on diffusion, KCl treatment failed to significantly elevate insulin levels outside of encapsulation in comparison to basal levels (p>0.9; FIG. 7C). In contrast, the application of a 30-second 11-kPa pressure led to a substantial increase in insulin levels outside the encapsulation (p<0.0001; FIG. 7C). These findings clearly demonstrated that KCl exposure effectively triggered insulin secretion from pseudoislets. However, achieving a corresponding elevation of insulin outside the encapsulation, on a physiologically relevant time scale, necessitated the application of a secondary driving force. These results align with previously reported observations of limited insulin release on a physiological time scale from encapsulated, electrically stimulated cells.

To test our second hypothesis, we compared insulin efflux driven solely by diffusion against that facilitated by brief pressure applied to encapsulated pseudoislets after 8 hours of islet encapsulation. A 30-second 11-kPa pressure application resulted in nearly 3 orders of magnitude higher insulin flux compared to diffusion alone (FIG. 7D). This unequivocally demonstrated the ability of brief pressure to enable rapid bolus insulin delivery within a sub-minute timeframe, irrespective of cell stimulation.

In assessing potential adverse effects of this brief pressure on β-cell health and basal secretion function, we administrated two additional pressure-driven doses at 8-hour intervals. Encouragingly, no decrement in insulin release was observed across consecutive doses (FIG. 7D). Moreover, our investigation into the viability of encapsulated primary human islets in vitro over a 5-day period revealed comparably preserved viability with both diffusion and pressure-based insulin delivery methods. The applied pressure level aligns with physiological norms and is maintained for only a very short duration, resulting in a slow flux and ensuring safety.

Taken together, these findings indicate that a brief application of pressure facilitates repeated and consistent bolus insulin delivery independent of beta-cell stimulation. Importantly, this method does not negatively impact the function of R7T1 pseudoislets or primary human islets. Such a property holds significant advantages for the advancement of cell-based therapies, as it circumvents previously (qualitatively) described insulin transport delays.

In vivo validation of insulin bolus delivery using wild type mice.

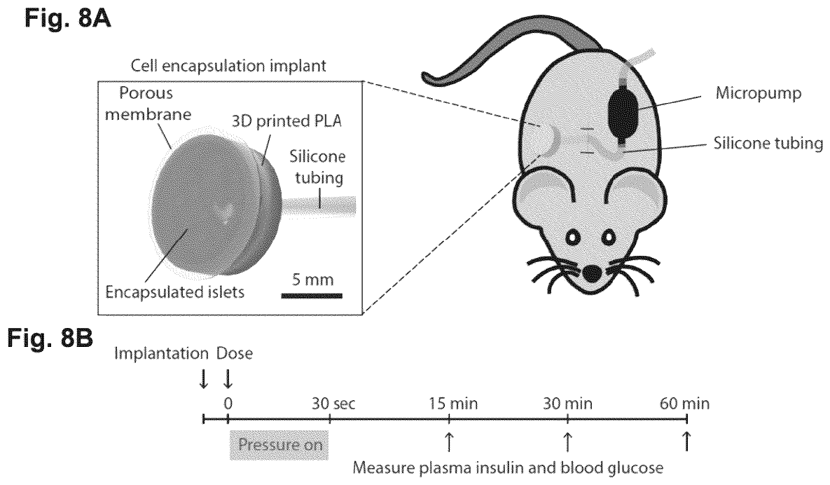

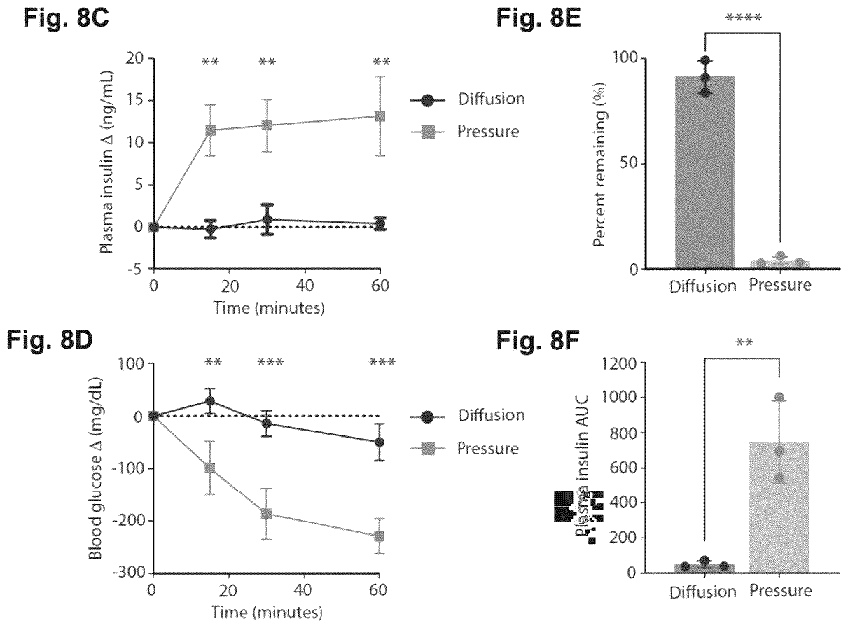

After confirming our findings through in vitro validation, we proceeded to conduct a comparative in vivo analysis. Subcutaneous implantation of encapsulated mouse islets was performed in wild type mice (C57BL/6J) (FIG. 8A). The implants used in all in vivo experiments elicited an anticipated foreign body response at two weeks.

Pressure-based dosing was performed immediately after device implantation. Following anesthesia, the average starting blood glucose was 286 mg/dL. To ensure accurate assessment of glucose effectiveness, we monitored both blood insulin and blood glucose levels post-implantation and dosing (FIG. 8b). A 30-second application of approximately 11-kPa pressure was employed for insulin bolus delivery from a 200-μL implant. Blood insulin levels in mice subjected to pressure-driven dosing were 35-fold higher than those treated with diffusion after 30 minutes (FIG. 8c). Consequently, blood glucose levels decreased by an average of 229 mg/dL with pressure driven dosing compared to 47 mg/dL with diffusion at 60 minutes (FIG. 4d). Analysis of the encapsulation device revealed that approximately 90% of the insulin secreted by the mouse islets remained within the device following 90 minutes of diffusion-based delivery, whereas less than 10% remained after pressure-driven delivery (FIG. 8e). Moreover, the blood insulin under the curve (AUC) was approximately 32-fold higher in animals receiving pressure-driven versus diffusion based delivery (FIG. 8f). These results demonstrate the necessity of pressure to generate a physiologically relevant insulin bolus in vivo.

### Restoring Euglycemia in Diabetic Mice Using Subcutaneous Implant and Pressure-Driven Insulin Dosing

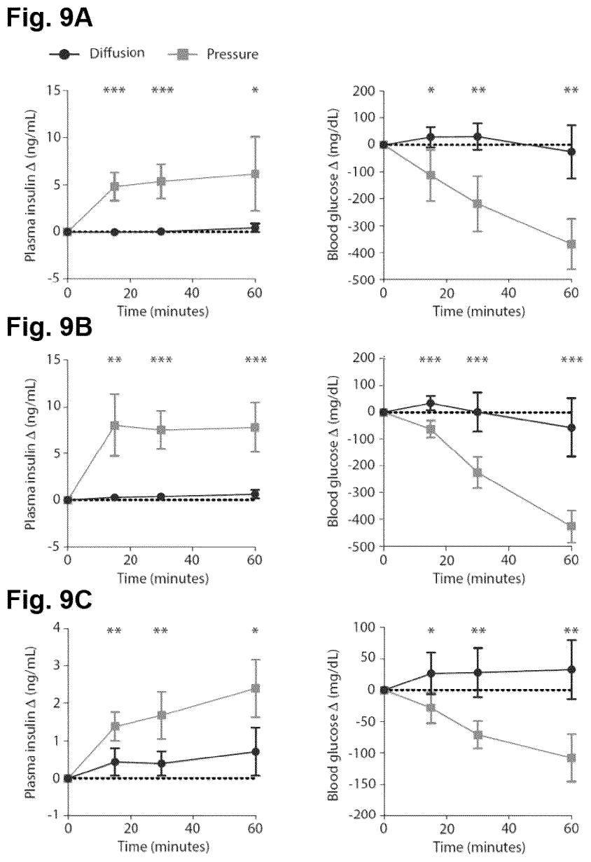

To assess the translational potential of pressure-driven insulin dosing, we conducted short-term in vivo transplantation experiments using encapsulated mouse and human islets in STZ-induced diabetic mice. Dosing was initiated immediately after device implantation. Mice dosed with applied pressure exhibited a significant increase in blood insulin levels, averaging 5.4 ng/ml for mouse islets and 7.6 ng/ml for human islets (xenogeneic transplantation) within 30 minutes. In contrast, animals treated with diffusion alone experienced a much lower increase in blood insulin levels during the same timeframe, averaging 0.04 ng/ml for mouse islets and 0.38 ng/ml for human islets. By 60 minutes, blood glucose levels decreased by an average of 368 mg/dL for mouse islets and 426 mg/dL for human islets with pressure-driven dosing, compared to an average decrease of 30 mg/dl and 57 mg/dL, respectively, with diffusion (FIG. 9a, 9b right). Notably, mice treated with pressure-driven dosing achieved euglycemia within 60 minutes, a result not achieved through diffusion-dependent insulin delivery from encapsulated islets. While diffusion alone did not achieve bolus delivery, a small impact on blood insulin was observed by 60 minutes (p=0.03 for human islets; FIG. 9b). These findings collectively demonstrated that applying brief pressure overcame limitations posed by diffusion, enabling efficient bolus insulin delivery.

Building on these promising results, we subsequently explored the potential of a renewable and scalable insulin source, namely reversibly transformed mouse R7T1 pseudoislets that can be proliferated as required, for managing blood glucose levels in STZ-induced hyperglycemic mice. Using pressure-driven dosing, we observed a significant reduction in blood glucose levels, with an average decrease of 108 mg/dl at 60 minutes. Conversely, when employing diffusion-based treatment, blood glucose levels actually increased by an average of 32 mg/dL, possibly due to glucose diffusion from the encapsulated cell media (FIG. 9c). This finding aligns with the modest glucose rise depicted in FIG. 8d. Our findings suggest that pressure-based dosing enhanced glucose control, offering the potential utility of a readily available biologic (R7T1 beta-cells) to traditional sources such as harvested mouse islets, human islets, or stem-cell-derived pancreatic cells.

Enhanced Glycemic Control in Diabetic Mice Using Repeated Pressure-Driven Insulin Delivery with Variable Durations and Intervals

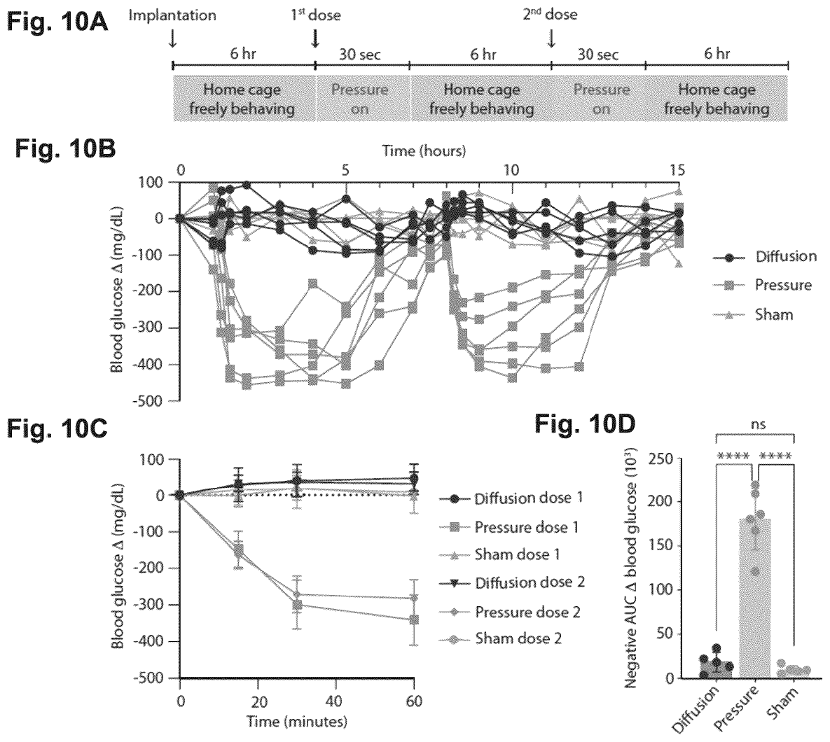

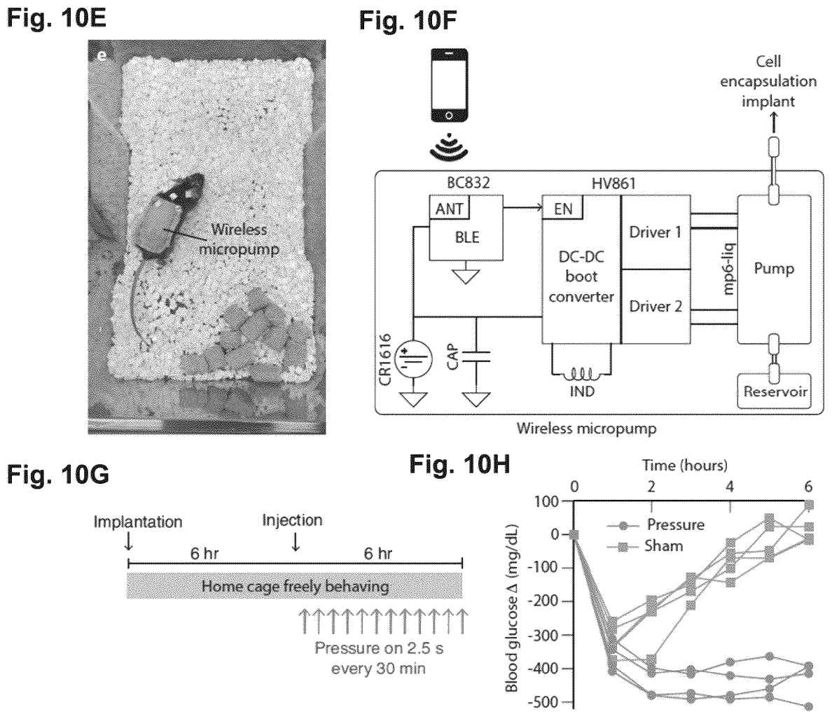

To ascertain the clinical viability of pressure-based insulin dosing, we examined its immediate impact on blood glucose regulation in diabetic mice via repeated bolus administrations (FIG. 10a). Approximately 400 mouse islets (˜480 IEQ, 1600 IEQ/cm2) were encapsulated in a 200-μL implant. Within 30 minutes of the initial pressure-driven bolus, there was a notable decrease in blood glucose levels, averaging 310 mg/dL (FIG. 10b). In contrast, mice receiving insulin through passive diffusion experienced an average increase of 41 mg/dL in blood glucose levels. Sham controls showed a modest decrease of 21 mg/dL, confirming that the significant drop in glucose observed in the experimental group was attributable to the administered insulin. The kinetics of this response closely matched the pharmacokinetic profile of subcutaneously injected regular insulin53. Subsequent administration of a second dose 6 hours later reaffirmed these results, with pressure delivery achieving a consistent glucose reduction of approximately 270 mg/dL, while diffusion resulted in a slight increase of 32 mg/dL (FIG. 10c). The total AUC for delta blood glucose significantly favored the pressure-treated group, showing a reduction to −180,560±35,003, compared to −18,549±11,338 for the diffusion-treated mice (FIG. 10d). Notably, the animals were free to behave naturally between doses, highlighting the method's robustness and reliability. By the end of the experiment, 6 hours post-final dose, all animals had returned to hyperglycemia (521±35 mg/dL), underscoring that the transient euglycemia was induced by the pressure-delivered doses and not by endogenous insulin production.

Next, we demonstrated the versatility of pressure-based insulin delivery via adjustable dosing duration and intervals through the development of a wireless, wearable micropump system. This system facilitates repeated pressure-based dosing in freely behaving mice, thereby eliminating the need for tethering or repeated anesthesia (FIG. 10e and Supplementary Movie 1). It comprises a piezoelectric micropump, a dual-channel pump driver, a Bluetooth (BLE) controller, and a 1-mL reservoir, all powered by a single coin-cell battery (FIG. 10f). To demonstrate utility, we reconfigured the 30-second dosing schedule from every 6 hour to even shorter 2.5-second intervals every 30 minutes, while keeping the total pressure duration unchanged (FIG. 10g). An initial subcutaneous insulin injection of 0.05 units was administered to lower blood glucose levels, followed by pressure-driven dosing. The total AUC for blood glucose with pressure-based dosing reached −2556, a significant improvement over the −804 recorded for controls (FIG. 10h; p<0.001). Compared to the above bolus delivery, smaller more frequent pressure-based dosing also resulted in a substantial reduction in blood glucose AUC, demonstrating short-term utility for maintaining euglycemia in mice with severe diabetes.

Beta-Cells as a Cell Factory for on-Demand Recombinant Protein Dosing: Harnessing the Neuroendocrine Cell Secretory Pathway for Controlled Release

Here, we propose harnessing the potential of beta cells, specifically R7T1 β-cell pseudoislets, as an on-demand cell factory for recombinant proteins. Beta cells stand out as an ideal target cell line due to their possession of peptide processing machinery, vesicle protein storage and a regulated vesicular fusion (cargo release). The proteins, once translated and processed, are stored within vesicles within the cells (Burgess and Kelly, 1987) (FIG. 2). The process of membrane depolarization, induced by stimuli such as glucose, KCl (FIG. 2), or electrical signals, propels the exocytosis of the stored protein from the cell. This intricate mechanism facilitates the prompt and controlled release of a substantial quantity of stored protein, responding swiftly to demand within a sub-minute to minute time frame. Furthermore, R7T1 β cells exhibit reversible immortalization and the capability to organize into pseudoislet clusters, thereby providing a mechanism for manipulating cell numbers and cell density within transplanted encapsulation. Furthermore, electrical coupling of R7T1 β-cell pseudoislets provides a mechanism for synchronized protein release. In this work, we demonstrate the use of β-cells for the synthesis of glucagon and GLP-1, highlighting their controlled release along the secretory release pathway of β-cells.

Beta cells natively produce and process insulin. Insulin is initially translated as preproinsulin, comprising a signal peptide (SP), A-chain, B-chain and C-peptide (FIG. 3A). The signal peptide guides preproinsulin into the endoplasmic reticulum for processing. The interaction of the signal peptide with cytosolic ribonucleoprotein signal recognition particles enables translocation into the lumen across the rough endoplasmic reticulum (Fu et al., 2013). Subsequently, the signal peptide is cleaved by signal peptidase, resulting in the formation of proinsulin (FIG. 3A). Proinsulin undergoes folding, forming three disulfide bonds, and is then transported to the Golgi apparatus. Within the Golgi apparatus, it enters secretory vesicles, where enzymes cleave proinsulin into mature insulin and C-peptide (FIG. 3A). Both mature insulin, composed of the A-chain and B-chain linked by disulfide bonds, and C-peptide are stored in vesicles within the cell.

To direct the release of the protein of interest by β-cells, we designed DNA vectors in which the C-peptide portion of proinsulin was substituted with our protein of interest. Additionally, mutations were introduced into the A-chain and B-chain of insulin to avoid co-secretion of bio-active insulin with the protein of interest. Additionally, we used CRISPR to disrupt genomically encoded insulin to prevent endogenous insulin production. Finally, using a luciferase-based expression platform, we validated that the engineered vectors follow the secretory pathway upon release.

### CRISPR-Mediated Insulin Knockout in R7T1 β-Cells

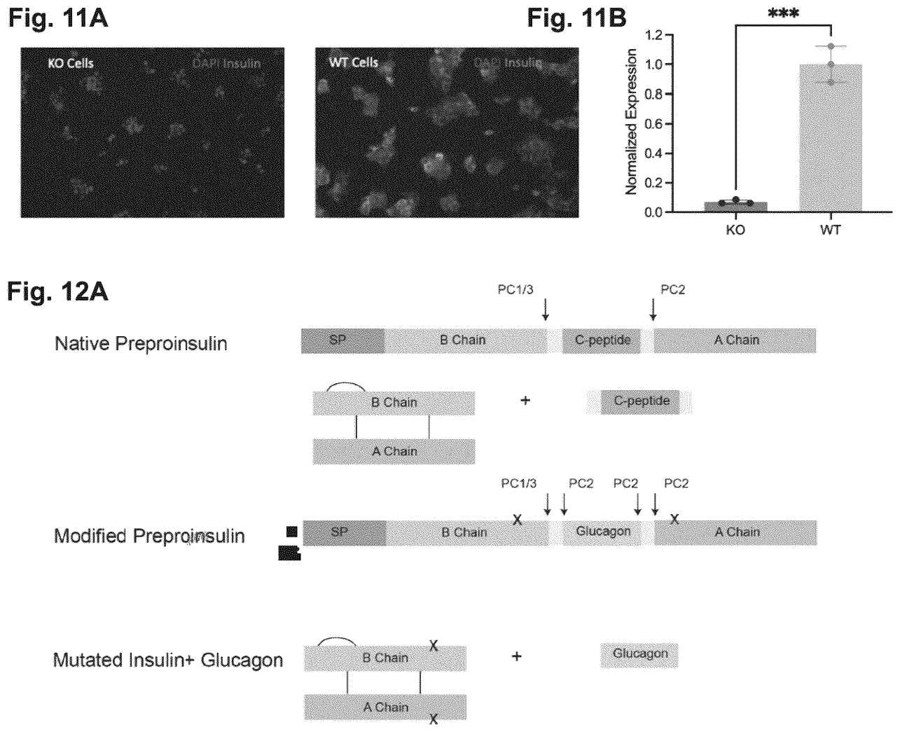

Utilizing CRISPR Cas9, we generated a line of insulin knockout R7T1-Cas9 β-cells to prevent cosecretion of insulin and target peptides in subsequent experiments. Following transduction of R7T1-Cas9 β-cells with INS1- and INS2-targeted guides, antibiotic selection (puromycin) and flow cytometry sorting (mCherry selection for transduced cells), we evaluated insulin expression levels in wild-type and knockout R7T1 β cells by immunostaining and ELISA. Immunostaining of knockout R7T1 β cells revealed a substantially reduced insulin-positive cell population compared to wild-type cells (FIG. 11A). Concordantly, knockout R7T1 β cells demonstrated substantially reduced insulin expression compared to wild-typ R7T1 cells as determined by ELISA (FIG. 11B). These data confirmed successful impairment of insulin expression in the targeted R7T1-Cas9 β-cells.

### Engineered Proinsulin Vector for Glucagon Secretion

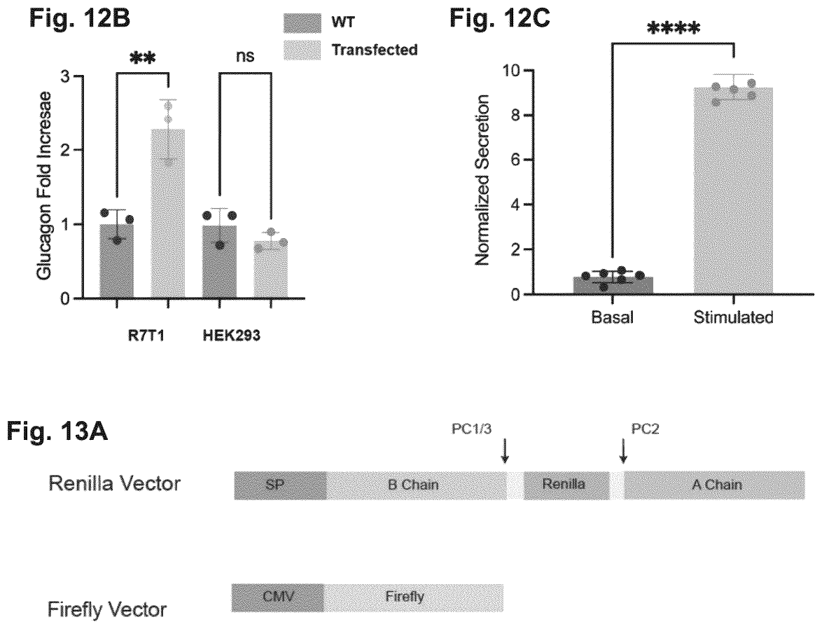

To harness the insulin processing capabilities of beta cells (FIG. 3A), we engineered the proinsulin vector to replace the C-peptide sequence with the glucagon sequence. In native preproinsulin, proinsulin undergoes processing to insulin through the enzymes prohormone convertase 2 (PC2) and prohormone convertase 1/3 (PC1/3) (FIG. 12A). Additional enzyme cleavage sites for PC2 are incorporated on both sides of the glucagon sequence to ensure the cleavage of additional amino acids, thereby eliminating any remaining linkers attached to the glucagon peptide (FIG. 12A). To prevent the co-secretion of active insulin and glucagon, two mutations were introduced to the A-chain and B-chain of insulin, rendering it a nonbioactive form. Consequently, the final vector was meticulously designed to co-secrete an inactive form of insulin alongside an active form of glucagon (FIG. 12A).

Using the engineered proinsulin vector, we assessed the secretion of glucagon in transfected R7T1 β cells and compared to control cells. Transfected cells exhibited a heightened level of glucagon secretion compared to untransfected controls (FIG. 12B). Despite the introduction of insulin mutations resulting in decreased secreted insulin levels, it had no impact on the secretion levels of our protein of interest. Additionally, HEK293 were transfected with the same engineered proinsulin vector, and in comparison, to control cells, no increased glucagon levels were detected in the conditioned media (FIG. 12B). This outcome suggests that the engineered proinsulin vector is undergoing processing and cleavage as intended, given that HEK293 cells lack the necessary enzymes (PC1/3 and PC2) to process proinsulin. Upon cell stimulation via KCl-driven membrane depolarization, secreted glucagon levels were nearly 8-fold higher than in unstimulated controls (FIG. 12C). This finding suggests that stimulation can effectively trigger the release of a bolus of glucagon from our genetically engineered cell line, indicating that glucagon is likely being released along the beta cell secretory pathway.

Validation of Constitutive Vs. Secretory Release Pathways

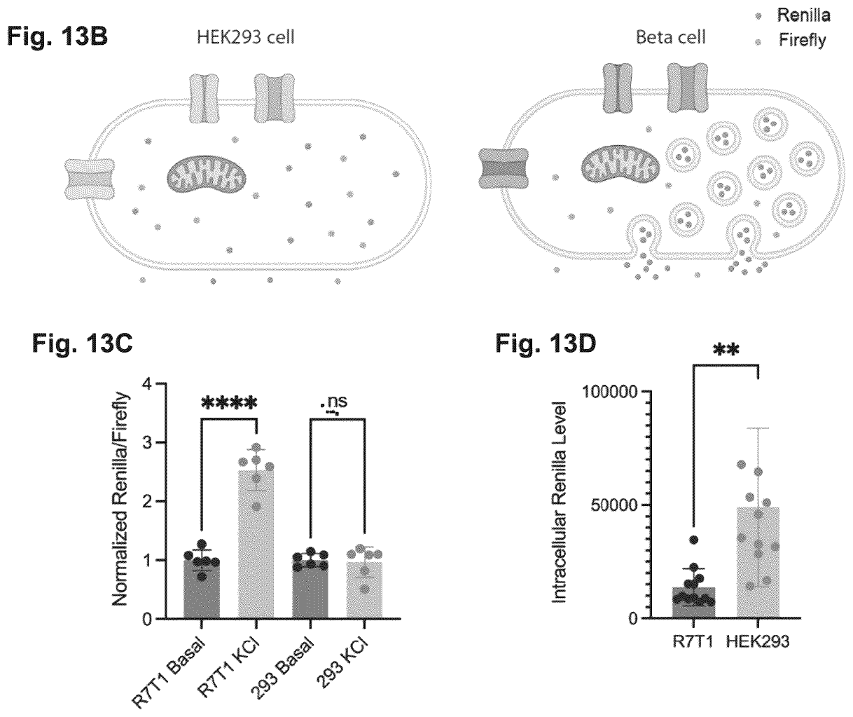

To validate the release pathways of the engineered proinsulin vectors, R7T1 β cells and HEK293 cells were co-transfected with two luciferase vectors: the proinsulin renilla and a CMV firefly (FIG. 13A). The signal peptide in the proinsulin renilla vector should direct the renilla luciferase along the secretory pathway, while the firefly vector lacks a signal peptide and should be directed to the constitutive pathway only. Additionally, the firefly expression level serves to normalize transfection efficiency between the various cell lines and conditions. Upon KCl stimulation, an increase in renilla luciferase secretion from R7T1 β cells is expected, but not from HEK293 cells (FIG. 13B).

In R7T1 β cells, KCl stimulation resulted in an increased secretion of renilla luciferase, normalized for transfection efficiency (FIG. 13C). Conversely, in HEK293 cells, KCl stimulation did not result in an increase secretion of renilla luciferase relative to basal levels, normalized for transfection efficiency (FIG. 13C). This indicates that the proinsulin vector is directed to the secretory pathway in the R7T1 β cells but is only constitutively released in the HEK293 cells. Importantly, the intracellular level of renilla luciferase was higher in HEK293 cells than in R7T1 β cells (FIG. 13D). This indicates that the difference observed in the previous experiment (FIG. 12B) is not due to the inability of HEK293 cells to translate the engineered proinsulin vector but rather due to the lack of granular protein storage in HEK293 cells.

### Utilizing the Engineered Proinsulin Vector as a Platform for Multiple Peptide Releases

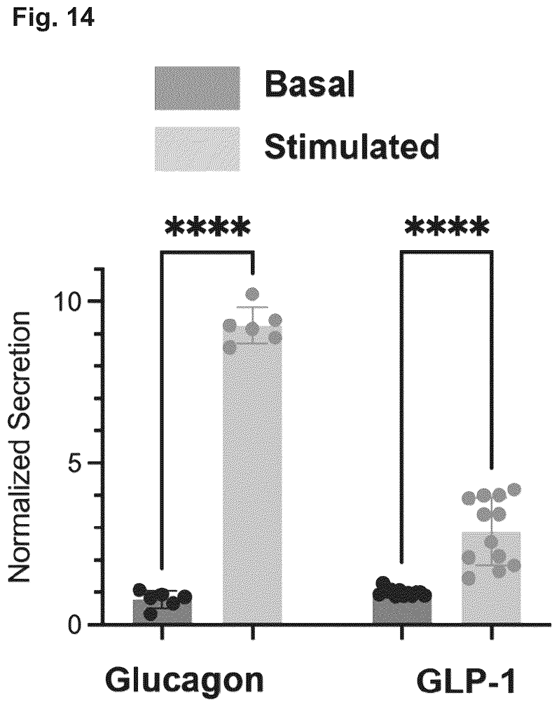

Beyond glucagon, we modified the proinsulin vector by substituting the C-peptide sequence with the GLP-1 sequence. Similar to the glucagon vector, we observed increased secretion of GLP-1 in transfected cells upon KCl stimulation (FIG. 14). This suggests that the engineered proinsulin vector is versatile and can be employed for the production of various peptides with stimulated secretion.

What is claimed is:

1. A system of proteinaceous species delivery, comprising:
biological secretory cells encapsulated within a porous membrane, wherein the biological secretory cells are configured to secrete a proteinaceous species; and
a means configured to provide a force to yield an efflux of secreted proteinaceous species out of the porous membrane.

2. The system of claim 1, wherein the force to yield an efflux of secreted proteinaceous species out of the porous membrane is an applied pressure.

3. The system of claim 2, wherein the means configured to provide the applied pressure is a micropump in operable connection with the porous membrane.

4. The system of claim 2, wherein the applied pressure is between 8 kPa and 15 kPa.

5. The system of claim 1, wherein the porous membrane is configured to allow efflux of the proteinaceous species and is configured to be immunoprotective.

6. The system of claim 1, wherein the porous membrane comprises a material selected from: poly(ethylene) (PE), poly(ethylene terephthalate) (PET), ultra-high molecular weight poly(ethylene) (UHMWPE), thermoplastic poly(urethane) (TPU), expanded poly(tetrafluoroethylene) (ePTFE), and alginate.

7. The system of claim 1, wherein the porous membrane has a thickness between 1 μm and 100 μm.

8. The system of claim 1, wherein the porous membrane has a porosity between 5% and 25% and has an average length of pore diameter between 5 μm and 2 mm.

9. The system of claim 1, wherein the biological secretory cells are characterized as comprising secretory granules that comprise the proteinaceous species that have been expressed and processed by the cell.

10. The system of claim 10, wherein depolarization of the biological secretory cell results in secretion of the proteinaceous species via the secretory granules.

11. The system of claim 1, wherein the biological cells comprise one or more of: endocrine cells, acinar cells, neuroendocrine cells, neurons, astrocytes, or granulocytes.

12. The system of claim 1, wherein the biological cells comprise one or more of: alpha (α) cells, beta (β) cells, pancreatic polypeptide (pp) cells, delta (δ) cells, epsilon (ε) cells, enterochromaffin (EC) cells, enterochromaffin-like (ECL) cells, gastrin (G) cells, cholecystokinin (I) cells, K cells, L cells, motilin (Mo) cells, neurotensin (N) cells, secretin(S) cells.

13. The system of claim 1, wherein the biological cells comprise beta (β) cells.

14. The system of claim 1, wherein the biological cells are provided within a microenvironment with multiple cell types.

15. The system of claim 1, wherein the proteinaceous species comprises one or more of: a hormone, a neuropeptide, a cytokine, an immunomodulator, a protein replacement, or an antigen binding species.

16. The system of claim 1, wherein the proteinaceous species comprises one or more of: insulin (INS), glucagon (GCG), gastric inhibitory polypeptide (GIP), glucagon-like peptide 1 (GLP-1), glucagon-like peptide 2 (GLP-2), leptin (LEP), pancreatic polypeptide (PP), somatostatin (SST), substance P (SP), ghrelin appetite hormone (GAH), gastrin (GAST), peptide YY (PYY), neuropeptide Y (NPY), cholecystokinin (CKK), angiotensin (Ang), neurotensin (NTS), islet amyloid polypeptide (IAPP), motilin (MLN), secretin (Sct), vasopressin (AVP), vasoactive intestinal peptide (VIP), calcitonin (CT), growth hormone (GH), insulin-like growth factor 1 (IGF-1), follicle-stimulating hormone (FSH), oxytocin (OT), thyroid-stimulating hormone (TSH), gonadotropin-releasing hormone 1 (GnRH1), gonadotropin-releasing hormone 2 (GnRH2), dynorphins (Dyn), endorphins, endomorphins (EM), nociceptin (NC). Examples of cytokines that can be expressed include (but are not limited to) type 1 interferons (IFNs), interferon gamma (IFNG), tumor necrosis factor alpha (TNF-α), interleukin 2 (IL-2), interleukin 7 (IL-7), interleukin 12 (IL-12), interleukin 21 (IL-21), programmed cell death protein 1 antibody (anti-PD-1), programmed death ligand 1 antibody (anti-PD-L1), and tumor necrosis factor alpha antibody (anti-TNF-α), factor VIII (FVIII), cystic fibrosis transmembrane conductance regulator (CFTR), α-glucocerebrosidase, α-galactosidase, receptor tyrosine-protein kinase erbB-2 antibody (anti-HER2); vascular endothelial growth factor antibody (anti-VEGF), cluster of differentiation 20 antibody (anti-CD20), or pathogen neutralizing antibodies.

17. The system of claim 1, wherein the proteinaceous species comprises one or more of: insulin, glucagon, or GLP-1.

18. The system of claim 1, wherein the proteinaceous species is a transgene product, expressed from an expression cassette, wherein the transgene is expressed as a preprocessed proteinaceous product, wherein the biological secretory cells express the cellular components to process the transgene such that it be secreted.

19. The system of claim 18, wherein the transgene product is expressed from a preproinsulin cassette, wherein a sequence of the transgene replaces a sequence of C-peptide in the sequence of preproinsulin.

20. The system of claim 1, wherein the system of proteinaceous species delivery is part of an implant for installation in a recipient.
